(19) United States
(12) Patent Application Publication
(10) Pub. No.: US 2003/0060890 A1
(43) Pub. Date: Mar. 27, 2003

(54) Joint prosthesis and method for implantation

(76) Inventor: Imad Ed. Tarabishy, Spring Hill, FL (US)

Correspondence Address: William S. Van Royen, PETTIS & VAN ROYEN, P.A., Suite 700, 501 E. Kennedy Blvd., Tampa, FL, 33602-5200, US

(21) Appl. No.: 10/161,935

(22) Filed: Jun. 4, 2002

Related U.S. Application Data
(63) Continuation-in-part of application No. 09/961,662, filed on Sep. 24, 2001.

(51) Int. Cl. A61F 2/32; A61F 2/34

(52) U.S. Cl. 623/22.12; 623/22.15; 623/22.2; 623/22.21; 606/89

(57) ABSTRACT

A prosthesis for replacement of a ball and socket joint in the human body. The prosthetic components comprise a plurality of separate parts which may be inserted through a hole in the femur of the patient and assembled and attached to the prepared acetabulum of the hip bone to form a cup-shaped first shell. A cup is also passed through the hole in the femur and attached to the first shell to form the socket portion of the joint. A shaft having a first end with a ball formed thereon is inserted in the hole through the femur so that the ball engages the cup for movement therein.

### BACKGROUND OF THE INVENTION

1. Field of the Invention

The present invention relates to a device for total joint replacement, and more particularly to hip and shoulder joint prostheses that require less invasive surgery during installation than required by the current prostheses and methods for placement.

2. Description of the Prior Art

Arthroplasty, the restoration of normal joint motion, is frequently done by the insertion of metallic prostheses. Implant technology has improved over the last number of years and provides solutions to problems caused by injury, arthritis and other joint diseases. Frequently, the damage is sufficiently severe to require a total joint replacement. The prior art discloses numerous designs for total hip joint prosthetic devices.

Total hip joint replacements require interactive prosthetic femoral and acetabular components to emulate the ball-socket mechanism of a natural hip joint. When the supporting structure is weakened, particularly the femoral head and neck, a prosthetic femoral component with an extended shaft is implanted within the medullary cavity of the femur. Examples of this type total hip replacement prosthetic device are disclosed in U.S. Pat. No. 6,093,208 issued to Enrico Tian and U.S. Pat. No. 5,807,407 issued to England, et al. Many surgeons take this route, even when the underlying bone structure of the femoral head and neck is strong, under the theory that implantation of the shaft within the medullary cavity of the femur is required to obtain the necessary support for the prosthetic femoral head, as the femoral implant is under high stresses that can cause failure of “surface replacement” devices. Such failures frequently occur early in the patient's recovery, before the bonding of the bone to the metal surfaces of the prosthetic implant has occurred. However, the insertion of the prosthetic device with a long femoral shaft requires the resection of the femoral head and neck to obtain access to the longitudinal cavity within the femur. Such surgery is very stressful to the patient and increases the risk of infection. If the device fails, any further implantation of prosthetic devices becomes exceedingly difficult, as the supporting bone structure has already been appreciably reduced.

U.S. Pat. No. 5,800,558 to Gerald A. LaHaise, Sr., U.S. Pat. No. 5,133,764 to Pappas et al., and U.S. Pat. No. 4,846,841 to Indong Oh, disclose the “surface replacement” technique of a total replacement of a hip joint. “Surface replacement” is aimed at primarily providing replacement of the joint surfaces while preserving as much of the supporting bone structure as possible and preserving the integrity of the medullary cavity. Pappas et al. '764 and Oh '841 each disclose a version of a cap that is implanted over the resected head of the femur. LaHaise '558 discloses a more complex means for attaching the ball to the resected head of a femur. One advantage to the surface replacement type of total hip replacement, is that much of the femur is left intact, so that if the surface replacement method fails, it may be replaced with an intramedullary canal prosthetic component.

Each of the above patents disclose a generally solid metal acetabular cup that is fixed, usually by screws, to a prepared surface of the hip bone. An insert, a layer of plastic or metal is frequently attached to the acetabular cup, the insert being sized to receive the ball portion of the prosthetic joint that is attached to the femur.

Each of these prostheses mentioned above, are installed during lengthy, invasive, major surgery that requires surgically opening the hip area for full exposure and direct access to the hip joint. During surgery the head of the femur must be removed from the acetabular cup, for resection of the femur head or complete removal of the femur head and neck. This surgery comes at a high cost as it is complex, requiring extensive surgical support staff and operating room equipment.

Not withstanding the existence of such prior art prosthetic components and methods for attachment to the human body, it remains clear that there is a need for prosthetic components that may be inserted into the human body without a major incision gain direct access to the femur and hip bone.

### SUMMARY OF THE INVENTION

The present invention relates to a prosthesis and method for implantation of that prosthesis within the human body as the replacement for a ball joint. The apparatus comprises at least one segmented shell that is attachable to the acetabulum of the hip bone of a patient. The segmented shell comprises a plurality of separate parts which may be inserted through a hole in the femur of the patient and assembled and attached to the hip bone to form a cup-shaped first shell.

A cup whose exterior is sized and configured to be received tightly within the interior of the first shell is also passed through the hole in the femur and then attached to the first shell. The interior of the cup is sized and configured to receive the ball portion of the prosthetic component.

A shaft having a first end with a ball formed thereon, that is sized and configured to be received within the cup is inserted within the hole through the femur so that the ball engages the cup for movement therein.

As the head of the femur and the acetabulum is larger than any hole that can be made through the neck of the femur, an expandable drill bit must be used to remove the head of the femur and a thin portion of the outer layer of the acetabulum.

The invention accordingly comprises an article of manufacturer possessing the features, properties, and the relation of elements which will be exemplified in the article hereinafter described, as well as the method for insertion of the article into the human body. The scope of the invention will be indicated in the claims.

### BRIEF DESCRIPTION OF THE DRAWINGS

For a fuller understanding of the nature and objects of the invention, reference should be had to the following detailed description taken in connection with the accompanying drawings, in which:

FIG. 1 is an isometric view of the hip joint prosthesis of this invention;

FIG. 2 is a cross-sectional elevational view taken along line 2-2 of FIG. 1;

FIG. 3 is a top plan view of the segmented first shell of the prosthesis of FIG. 1;

FIG. 4 is an exploded cross-sectional view of FIG. 3 taken along line 4-4;

FIG. 4A is a detailed view illustrating the use of surgical thread in the place of wires 30;

FIG. 5 is an isometric view of the segmented first shell of FIG. 3 with the key segment exploded;

FIG. 6 is a top plan view of the segmented second shell of this invention with a portion cutaway in cross-section to illustrate the relationship of the parts;

FIG. 7 is a cross-sectional elevational view taken along line 7-7 of FIG. 6.

FIG. 8 is a detailed front elevational view of one of the second group of parts of the segmented shell of this invention;

FIG. 9 is a cross-sectional view taken along line 9-9 of FIG. 8;

FIG. 10 is a side elevational view of one of the first group of parts of the segmented second shell of FIG. 6;

FIG. 11 is a front elevational view of one of the first group of parts of the segmented second shell of this invention;

FIG. 12 is an isometric view of the shaft and cup of this invention;

FIG. 13 is a bottom plan view of the shaft of FIG. 12;

FIG. 14 is an isometric view illustrating a second preferred embodiment of the hip joint prosthesis of this invention;

FIG. 15 is an isometric view illustrating a third preferred embodiment of the hip joint prosthesis of this invention;

FIG. 16 is a detailed view of the hip structure illustrating the placement of the guide wire into the hip bone;

FIG. 17 illustrates the placement of a protective hollow blunt guide and the hole bored through the neck and head of the femur and into the acetabulum of the hip bone;

FIG. 18 illustrates the placement of a sleeve into the hole bored through the femur;

FIG. 19 illustrates the insertion of the expandable bit into the sleeve;

FIG. 20 illustrates the extension and expansion of the expandable bit for removal of a portion of the acetabulum of the hip bone;

FIG. 21 illustrates the placement of a segmented first shell and transportation of one of the plurality of segments through the sleeve guided by a secondary guide wire for placement onto the base and engagement with an adjacent segment;

FIG. 22 illustrates the placement of a segmented first shell of this invention having thick segments;

FIG. 23 illustrates the placement of the cup within the segmented first shell of FIG. 22 and placement of the shaft into the hole bored through the femur so that the ball engages the cup;

FIG. 24 illustrates the placement of a first segmented shell having thin segments and a second segmented shell;

FIG. 25 illustrates the placement of the cup into the first and second segmented shells of FIG. 24;

FIG. 26 illustrates the placement of the shaft into the hole through the femur and engagement of the ball with the cup of FIG. 25;

FIG. 27 illustrates the placement of the segmented first shell, a solid second shell, a cup and the shaft that is inserted into the hole bored through the femur so that the ball engages the cup;

FIG. 28 is a front elevational view of the expendable drill bit used to remove the head of the femur, illustrating the blades extended outwardly;

FIG. 29 is a detailed view of the first end of the expandable drill bit of FIG. 28;

FIG. 30 is a detailed view of one of the blades of the cutter of FIG. 28;

FIG. 31 is a cross-sectional view taken along line 31-31 of FIG. 30;

FIG. 32 is a detailed top plan view of FIG. 28;

FIG. 33 is a detailed view of the means for attachment of the blades to the expandable drill bit of FIG. 28; and

FIG. 34 illustrates the attachment of the expandable drill bit to a drill motor for rotation of the expandable drill bit.

FIG. 35 is an isometric view of a fourth preferred embodiment of the hip joint prosthesis of this invention;

FIG. 36 is a cross-sectional elevational view taken along line 36-36 of FIG. 35;

FIG. 37 is a top plan view of the segmented shell of the prosthesis of FIG. 35;

FIG. 38 is a cross-sectional elevational view taken along line 38-38 of FIG. 37.

FIG. 38A is a detailed view illustrating the connection between the segments and the base and the partial insertion of the tooth into the niche to provide a segmented shell with the shortest diameter;

FIG. 38B is a detailed view illustrating the connection between the segments and the base and the full insertion of the tooth into the niche to provide a segmented shell with the largest diameter;

FIG. 38C is a schematic representation of a cross-section of the shell illustrating the expandability of the shell.

FIG. 39 is an isometric view of the segmented shell of FIG. 37;

FIG. 40 is a detailed isometric view of one of the segments of the invention of FIG. 35;

FIG. 41 is a detailed top plan view of the base of the segmented shell of this invention of FIG. 35;

FIG. 42 is a front elevational view of the base of FIG. 41;

FIG. 43 is a cross-sectional view taken along line 43-43 of FIG. 41;

FIG. 44 illustrates the placement of the base and transportation of two segments of the plurality of segments through the sleeve, being guided by a guide loop for placement into the base and engagement with an adjacent segment;

FIG. 45 illustrates the seating stage of the installation of the segments into the base;

FIG. 46 is a front elevational view of the segment tensioner of the invention of FIG. 35;

FIG. 47 is a top plan view of the loop holder of the tensioner of FIG. 46;

FIG. 48 is a front elevational view of the loop holder of FIG. 46.

FIG. 49 is a cross-sectional view of the loop holder taken along line 49-49 of FIG. 48;

FIG. 50 is a detailed view of FIG. 35;

FIG. 51 is a detailed view of FIG. 36;

FIG. 52 is a top plan view of a second embodiment of the segmented shell of the invention of FIG. 35;

FIG. 53 is a cross-sectional view taken along line 53-53 of FIG. 52;

FIG. 54 is an isometric view of the second embodiment of the segmented shell of FIG. 35;

FIG. 55 is a detailed view of the first, last, and next to last segments of the segmented shell of FIG. 52;

FIG. 56 illustrates the placement of the base with the fires segment and the second segment ready to be inserted; and

FIG. 57 illustrates the tensioning of the next to last segment upon installation.

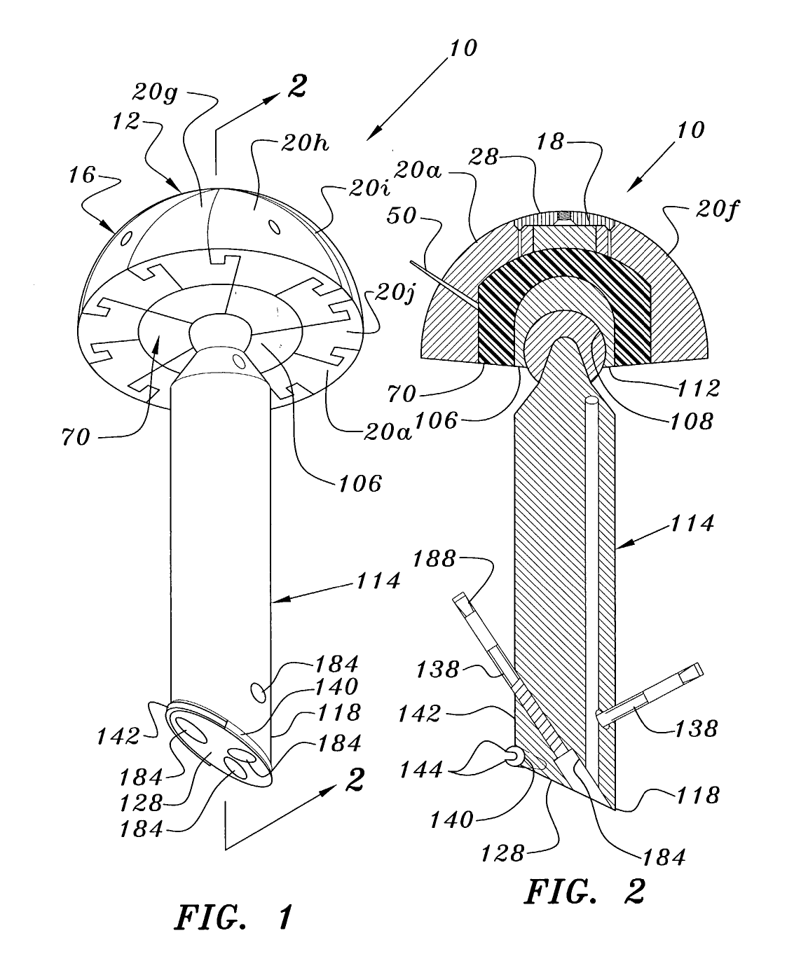

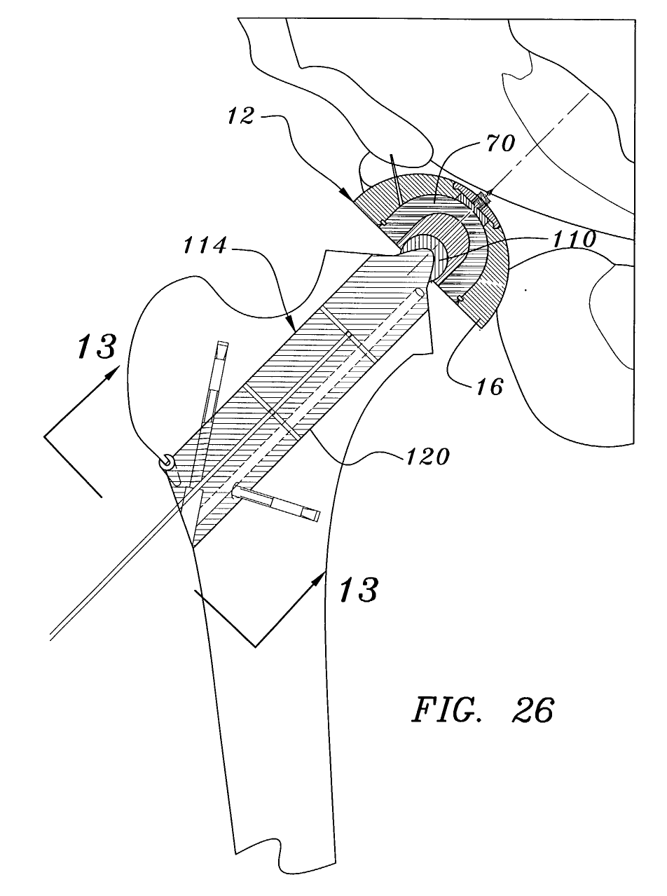

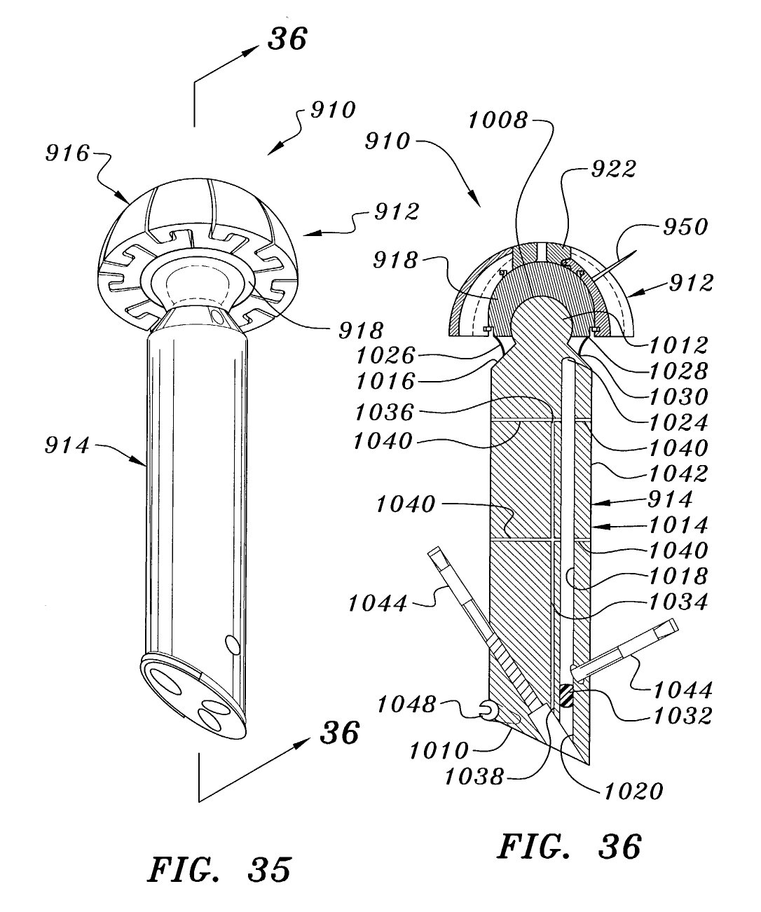

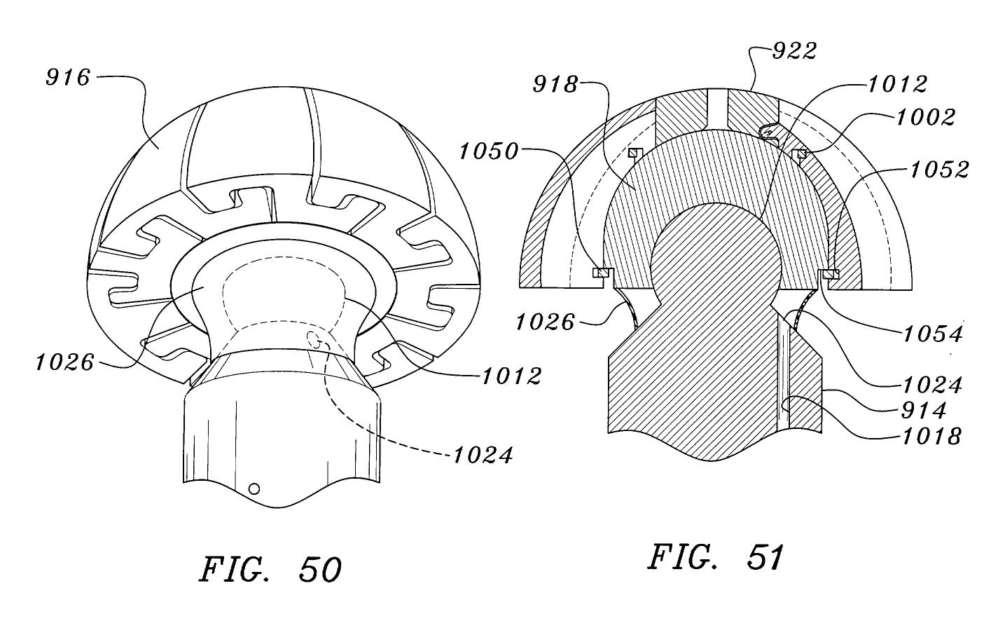

Similar reference characters refer to similar parts throughout the several views of the drawings FIGS. 1-34 and will be increased by increments of 300. Similar reference characters in FIGS. 35-52 will not necessarily refer to similar parts in drawing FIGS. 1-34; however, the reference characters will be increased by an increment of 900 over the first embodiment. The reference characters for similar parts in drawings 35-52 for an additional embodiment of the socket implant is increased by an increment of 300.

### DESCRIPTION OF A PREFERRED EMBODIMENT

A preferred embodiment for the prosthetic components of this invention are illustrated in the drawing FIGS. 1-13, in which the apparatus is generally indicated as 10. The prosthetic implants for replacement of a natural ball and socket joint and the method of placement of such components, as disclosed in this application, particularly illustrate a total hip joint replacement; however based upon the teachings of this patent, those skilled in the art will be able to modify these prosthetic implants to serve as a total shoulder joint replacement. Those skilled in the art will also be able to modify the steps for placement of a hip prosthesis, as taught by this patent, to implant a shoulder prosthesis. Referring first to the view of FIG. 1, it can be seen that the prosthetic components of this invention comprise a socket implant, shown generally as 12, and a ball implant, conveniently shaft 114. The ball implant is received by the socket implant to complete the mechanical joint for replacement of the natural joint.

For a total hip joint replacement, the acetabulum of the patient is prepared for receipt of the socket implant 12 and the femur is prepared for receipt of the ball implant. For a total shoulder joint replacement, the glenoid cavity of the scapula is prepared for receipt of a socket implant and the humorous bone is prepared for receipt of the ball implant. In the case of a shoulder joint, the portion of the socket implant that is attached directly to bone may be solid or segmented, as the shoulder socket implant is much smaller than the hip socket implant, and the size of the humorous bone in comparison with the femur supports a larger access bore for passing a solid shoulder socket implants therethrough. In both cases, the natural ball and socket joint is replaced with a mechanical one.

For purposes of illustration, the hip joint will be used to illustrate the apparatus and method of implantation. A preferred embodiment of the socket implant 12 is illustrated in the FIGS. 1-13, and comprises a first segmented shell 16 made of metal, a second segmented shell 70 made from synthetic resin, and a metal cup 106. In other preferred embodiments, structure of the socket implant 12 of the prosthetic components 10 will be modified according to the need, to form two additional embodiments, 310 and 610.

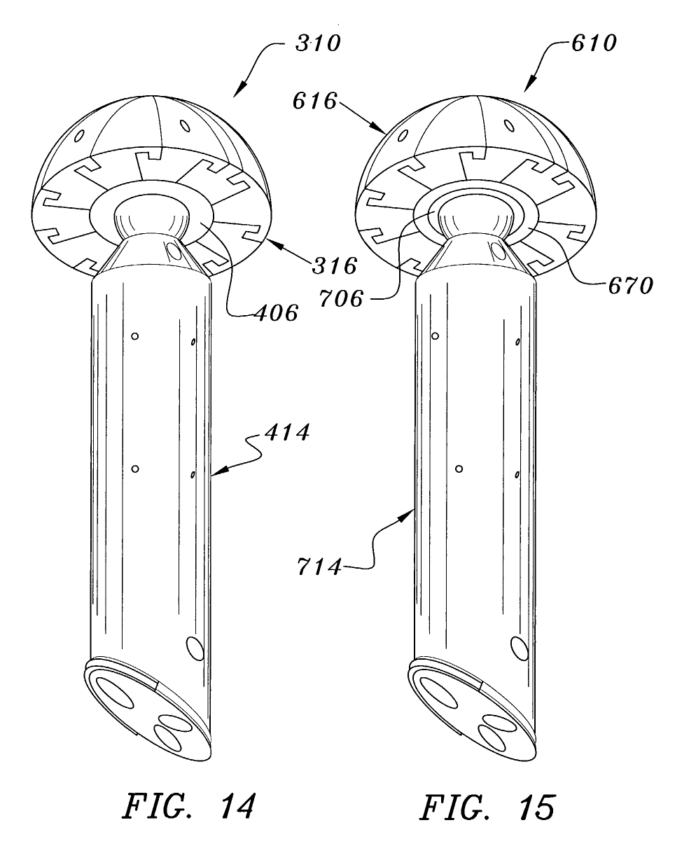

In a second preferred embodiment of the prosthetic components, indicated as 310, and as illustrated in FIGS. 14, 22, and 23, the socket implant 312 is comprised of a segmented metal first shell 316 and a cup 406 that is received by the first shell, the cup being made from metal or plastic. In order to use a single segmented shell and a cup, the patient receiving the prosthetic components 310 must have large bones so that the hole bored through the neck of the femur is large enough to allow thicker segments to pass therethrough and/or a greater number of thicker segments.

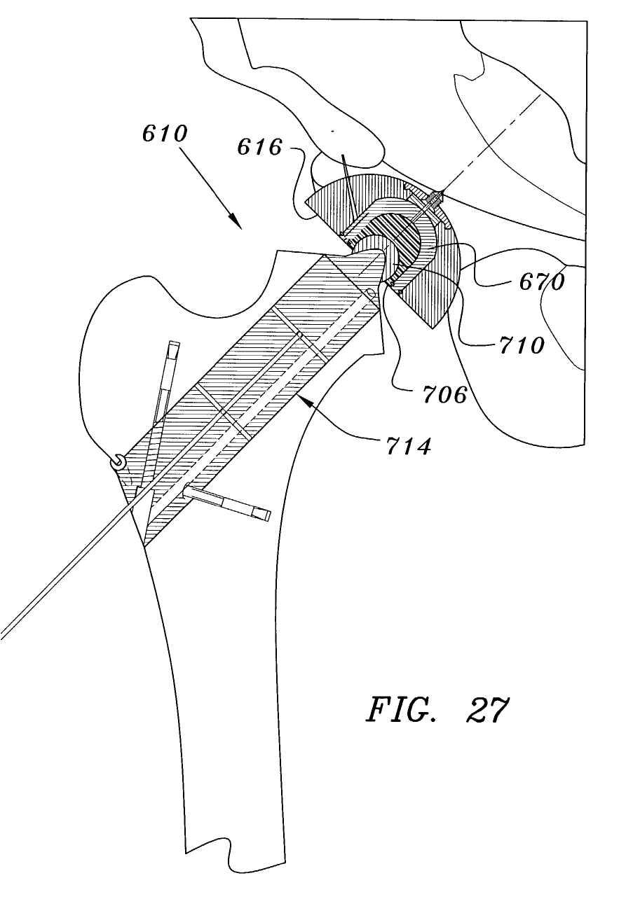

In third preferred embodiment of the prosthetic components, indicated as 610, and as illustrated in FIGS. 15 and 27, the socket implant 612 is comprised of a metal segmented first shell 616, which receives a second shell 670 that is constructed from a single piece of metal, which then receives a synthetic resin cup 706. Again, the bore through the femur must be large enough to receive the single piece metal second shell.

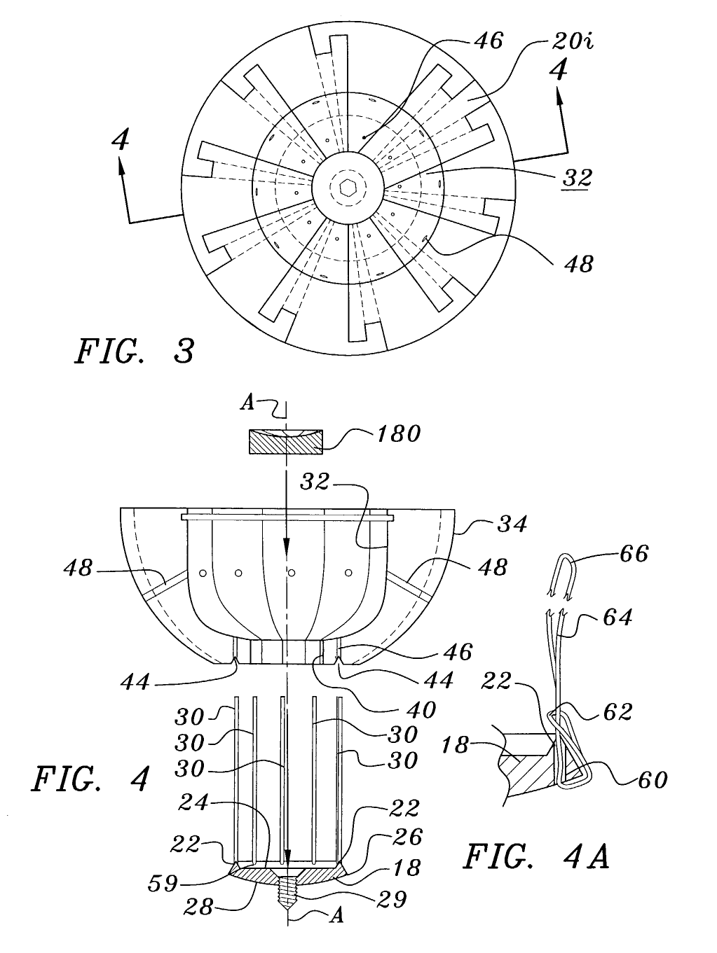

As it is the most complex, the primary discussion will be directed to the preferred embodiment of the prosthetic components 10, as illustrated in FIGS. 1-13. This does not mean that this is an embodiment that is preferred over the other embodiments, as embodiments that are less complex are preferred, if the patient's bone structure is capable of receiving it. The socket implant 12 comprises a segmented first shell 16, which will be mounted into the prepared hip acetabulum. The segmented first shell 16 is comprised of a base 18 and a plurality of segments 20a-j that engage one another. The term segment is defined as a separate piece of something and segmented is defined as something that is comprised of separate pieces. Any convenient number of segments can be used; however, ten segments have been used for illustration purposes. Therefore the segmented first shell 16 is comprised of separate pieces or segments 20a-j that are joined together to form the segmented first shell 16. As seen more clearly in FIG. 4, the base 18 has at least one ridge 22 that is formed in the top surface 24 of the base 18 proximal to the edge 26 of the base 18 and projects outwardly therefrom. In a preferred embodiment, the ridge 22 is continuous; however, this is unnecessary as long as a portion of the ridge 22 engages each segment 20a-j. The base 18 has a hole 27 therethrough for receipt of a cannulated screw 29 for attachment of the base 18 to the hip bone. The bottom surface 28 of the base 18 is curved to fit the curvature of the prepared surface on the hip bone. A plurality of flexible secondary guide wires 30, one for each segment 20a-j, are threadably mounted to the ridge 22 so that they are spaced apart from one another.

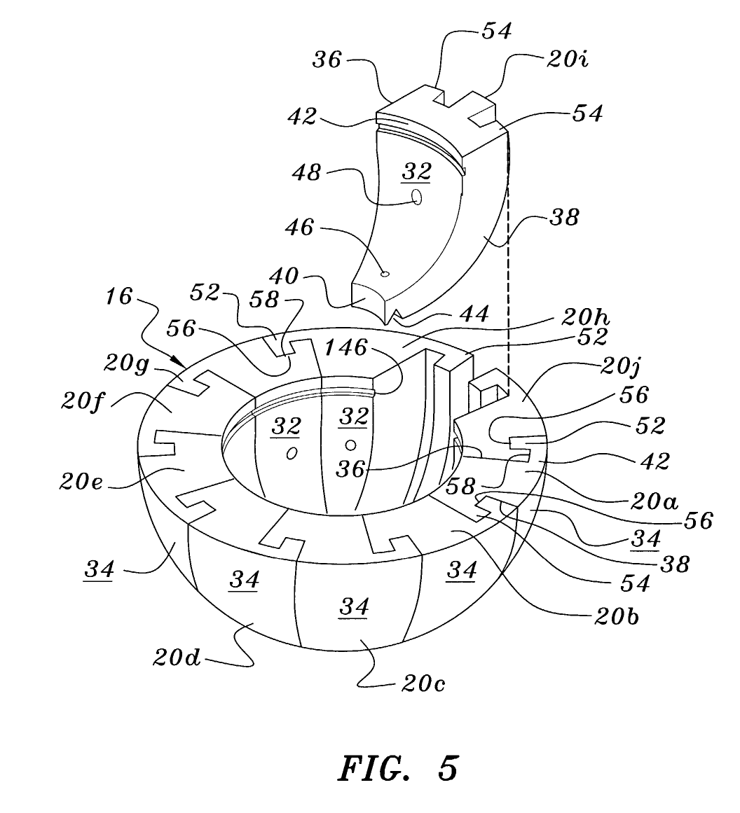

Each segment 20a-j has an inner surface 32, an outer surface 34, a longitudinal first side 36, a longitudinal second side 38, a first end 40 and a second end 42. The outer surface 34 of each segment 20 is longitudinally and transversely arcuate, with a curvature that will match the curvature of the prepared surface of the hip. Each segment has a groove 44 formed in the outer surface 34 proximal the first end 40, as seen in FIG. 3 and FIG. 5, and a hole 46 that extends from the bottom of the groove to the inner surface 32. Two or more of the segments also have a hole 48 that extends through the inner and outer surfaces of the segment intermediate the first and second ends 40 and 42 of the segment. At least two of the holes 48 receive surgical nails 50 therethrough, one nail 50 being visible in FIG. 2, that are used to attach some of the segments 20a-j to the prepared bone surface.

As seen most clearly in FIG. 5, all but two of the segments 20a-j have the same interlocking lands and grooves which can be described as male and female. The segments 20a-g are identical each having a male arm 52 that extends outwardly from the first side 36 and then inwardly toward the longitudinal axis A of the shell 16. The arm 52 extends longitudinally from the second end 42 toward and proximal to the first end 40. A female second arm 54 extends outwardly from the second side 38 of each of the segments 20a-j and then away from the longitudinal axis A of the shell 16 to form a groove 58. The groove 58 is sized and configured to receive the land 56 of the male arm 52 to form a land and groove interlocking connection. Segment 20i is the last segment to be attached to adjoining segments, and therefore must be inserted from the inside as the shell is generally spherical in shape, and thus the interior of the shell has a smaller circumference than the exterior. Therefore segment 20i must be wedge shaped with the outer surface 34 being narrower than the inner surface 32. The adjacent sides of the two adjoining segments, 20h and 20j, also must be modified to receive the wedge shape of the segment 20i. In addition, segment 20i must have two female second arms 54 and the adjacent segment 20h must have two male first arms 52 to enable the last segment 20i to interlock with the adjacent segments completing the shell 16.

In a preferred embodiment of prosthetic components 10, the first shell 16 is constructed from titanium or cobalt chrome. In other preferred embodiments of prosthetic components 10, other materials that are very resistant to wear are suitable for the purpose.

In a preferred embodiment of prosthetic components 10, the shell 16 will be received by the surgeon in disassembled form for thorough disinfecting prior to assembly in the patient by the surgeon. During assembly of the first shell 16, each segment is mounted on a respective secondary guide wire 30 and advanced downwardly until the groove 44 engages the ridge 22 on the base 18. The secondary guide wires 30 guide their corresponding segments to their proper position. These wires must be very flexible to enable the segments to easily slide down the wire in the cramped space used during the surgery, to be described in greater detail below. After assembly of the shell, the flexible wires 30 are unscrewed from their threaded holes 59 and then removed. In another preferred embodiment, holes are bored all the way through the base 18 to provide a new hole 60. As seen in FIG. 4A, the first end 62 of a closed loop 64 of surgical thread, is passed through the hole 60 in the base 18 from the top downwardly. The second end 66 of the loop is then passed through the loop at the first end 62 to connect the thread to the base 18. The first end of the thread loop 66 is then passed through the hole 46 in one of the segments 20a-j. Once the segments 20a-j are in place, each of the plurality of loops of thread 64 are cut and the thread 64 is pulled from the base 18 through the hole 46 in the segments 20a-j.

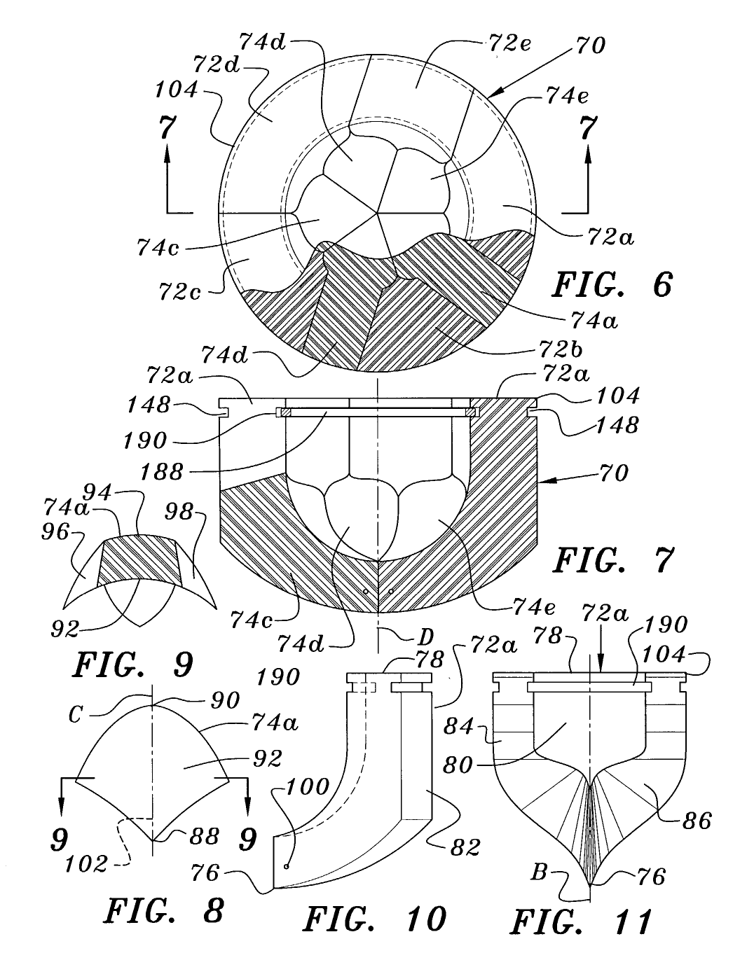

In a preferred embodiment of prosthetic components 10, a segmented second shell, shown generally as 70 in FIGS. 6-11, is sized and configured so that it can be assembled within the first segmented shell 16. The assembly will be discussed further below. The second shell 70 comprises a plurality of parts, those skilled in the art will easily be able to determine the suitable number of parts to be used for a particular patient. A total of 10 parts have been used for illustrative purposes, a first group of parts 72a-e and a second group of parts 74a-e. Each of the group of parts 72a-e have a longitudinal axis B, a first end 76, a second end 78, a first face 80, a second face 82, a first side 84 and a second side 86. Each of the second group of parts 74a-e have a longitudinal axis C, a first end 88, a second end 90, a first face 92, a second face 94, a first side 96, and a second side 98. The first face 80 and the second face 82 of the first group of parts and the first face 92 and the second face 94 of the second group of parts are each longitudinally and transversely arcuate. The first ends 76 and 88 of each part of the first and second group of parts is linked to one another, preferably by surgical thread passing through the hole 100 in the parts 72a-e and the hole 102 in the parts 74a-e, as seen in FIGS. 10 and 8 respectively.

A portion of the sides 84 and 86 of each of the parts 72a-d, that is proximal the outer rim 104 of the shell 70 extend radially toward the central axis D. When the parts 74a-e are being assembled to form the cup shaped shell 70, the last part 72e must be tapered outwardly so that the sides 84 and 86 are angled toward one another and thus the horizontal arc of the second face 82 is smaller than the horizontal arc of the first face 80. To ensure that all sides fit tightly, the sides of the adjacent parts, 72a and 72d, must be tapered inwardly to match the taper of part 72e so that the sides fit tightly when the parts are fully assembled.

The sides 84 and 86 of each of the parts 74a-d are tapered outwardly so that the first face 92 is much larger than the second face 94. The transverse dimensions of the second face 94 of the parts 74a-d vary along the longitudinal axis C between the first end 88 and the second end 90. The horizontal width of the second face 94 varies from a sharp edge (where the cross-section of the part would be triangular) proximal the first end 88 to a greater cross-section as seen in FIG. 9 and then narrows proximal the second end 90 to a generally triangular cross-section. As each of the parts 74a-e is inserted between adjoining parts 72a-e, as seen in FIG. 6, the curvatures of the adjacent sides must match one another to make a tight friction fit. The segmented second shell 70 may be constructed from synthetic resin or a suitable metal.

In a preferred embodiment of the prosthetic component 10, the segmented second shell 70 may be made using the same structure used for the first segmented shell 16, discussed above. This structure, using a base having threaded guide wires and interlocking segments will be just as satisfactory a structure as the second segmented shell 70 having a first group of parts 72a-e and a second group of parts 74a-e. The primary difference will be that the second shell will be smaller than the first shell and made from a suitable synthetic resin or metal. The second shell when made of metal will be attached to the first segmented shell 16 by snap rings, and when made of synthetic resin the second shell will be attached to the first segmented shell 16 by heat and pressure or by snap rings.

As shown in FIG. 2, a cup 106 is inserted within the second shell 70, which is preferably made of solid titanium or cobalt chrome with a very polished interior surface 108. The metal cup is used when the second shell 70 is constructed from synthetic resin. In other preferred embodiments of the prosthetic components 10, the cup 106 may be constructed from a synthetic resin, particularly when the second shell 70 is constructed from metal. When constructed from a synthetic resin, the cup 106 may be formed so that when the cup 106 receives the ball 110, the edge 112 of the cup 106 extends beyond the equator of the ball 110 so that the cup 106 snaps onto and is retained on the ball 110 for easy insertion into the socket implant 12. The cup 106 may be attached to the second shell 70 by a snap ring.

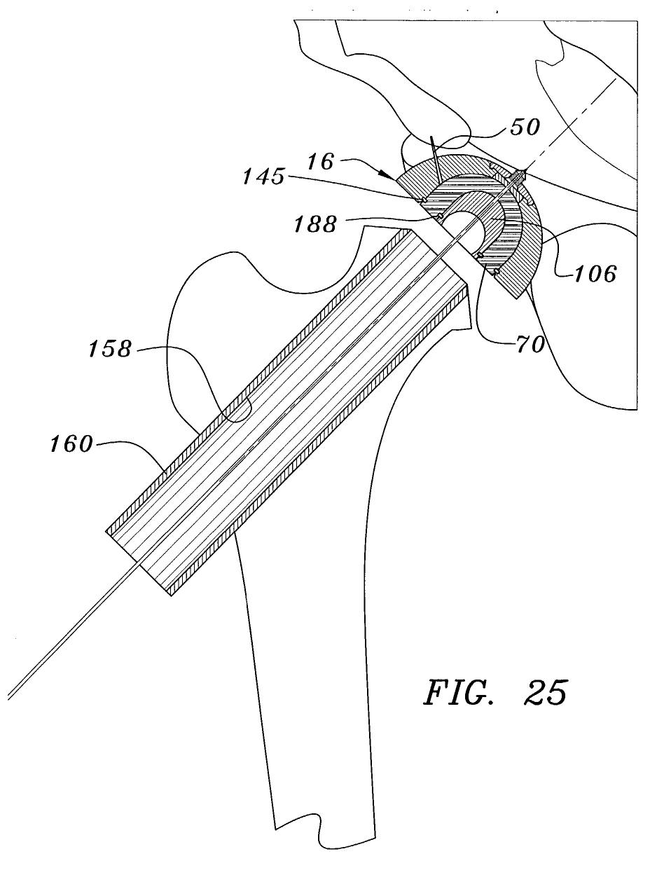

The shells and the cup may be attached to one another in a number of different ways. Snap rings or drop rings operate simply. Snap ring or drop ring 145, as seen in FIG. 25, is inserted within a groove 146, as seen in FIG. 5 that matches a second groove 148 in the second shell 70, as seen in FIG. 7. When the second shell 70 is pressed into place, the snap ring 145 is expands into the groove 146 so that when the groove 146 aligns with the groove 148 the snap ring shrinks into the groove 148 locking the two shells together. Snap ring 188 in groove 190, as seen in FIGS. 7 and 25, may engage the groove 182, as seen in FIG. 12, in the cup 106. When a plastic cup or plastic segmented shell is to be attached to an inner metal shell, heat (approximately 400 degrees) and pressure can be applied to the plastic cup or shell so that it bonds to the roughened surface of the metal shell. In other embodiments, it is possible to use well-known biologically tolerable bonding resins for that purpose.

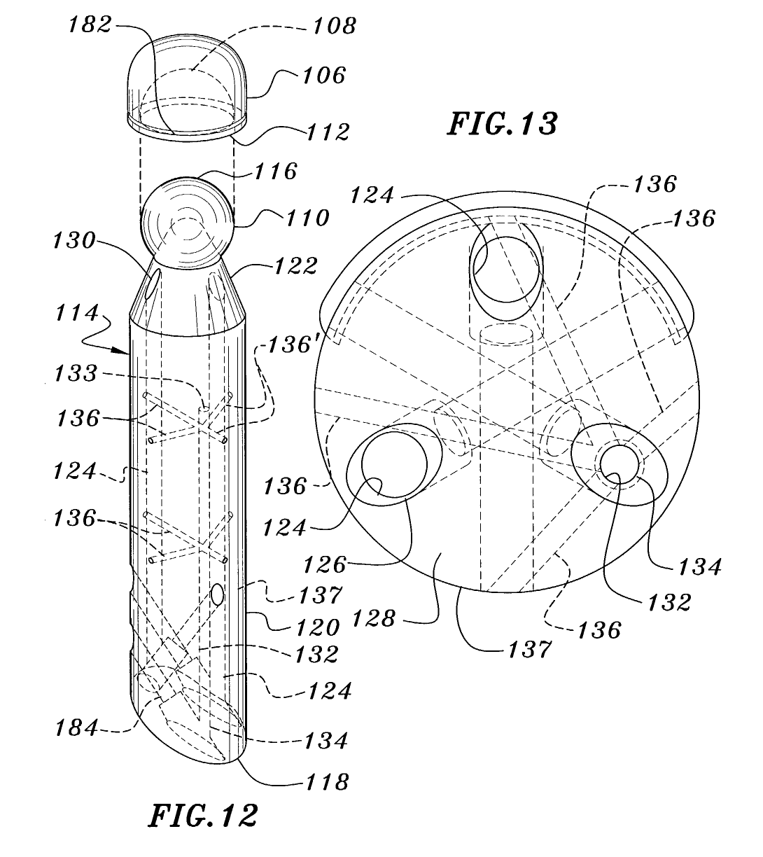

The ball implant 14, conveniently shaft 114, as shown in FIGS. 12 and 13, has a first end 116 and a second end 118. The shaft 114 is comprised of the ball 110, a body 120, and a neck 122 that attaches the ball 110 to the body 120. The ball 110 is generally spherical and is comprised of highly polished titanium or cobalt chrome. The neck 122 and the body are comprised of steel and the body 120 is coated with any one of the well-known ingrowth surfaces for encouraging the growth of the bone to that surface for bonding the body 120 to the adjacent bone of the femur. The shaft 114 is cannulated, having at least one tube 124 that extends from an open first end 126 at the bottom 128 of the shaft 114 to an open second end 130 at the neck 122. The tube 124 is used to flush and suction the site of the hip joint to remove debris that may have collected in the joint area due to the wear of the head 110 or cup 106 and may also be used to insert antibiotics to fight infection that may occur after implantation of the prosthetic components 10. A bag constructed from a formulation of silicon and rubber (or other material that is non toxic to the surrounding tissues) may be implanted in the patient's leg adjacent the second end 118 of the shaft 114 and attached by well known means to the shaft 114 so that it is in fluid flow communication with the tube or tubes 124. The bag will collect debris created by the prosthetic joint to prevent the debris from a toxic interaction with the surrounding tissue. An open second tube 132 also extends inwardly from the bottom of the shaft 114 to a closed second end 133 proximal the neck 122. The second tube 132 has an open first end 134 that is threaded for attachment of a syringe through which a cement may be injected into the tube 132 to bond the body 120 to the surrounding bone. The threads on the second tube 132 may also be used for attachment of an extraction tool (not shown) to remove the shaft 114, if that should be necessary. At least one secondary tube 136 is connected at one point along the second tube 132 and is in fluid flow communication therewith. The secondary tube 136 extends through the side wall 137 of the body 120 so that cement may be applied between the body 120 and the surrounding bone. For an even distribution of the cement around the body 120, a plurality of secondary tubes 136 interconnect with the second tube 132. For a more even distribution along the longitudinal length of the body 120, a second set of secondary tubes 136′ may interconnect in fluid flow communication with the tube 132 to apply cement closer to the neck 122. In the preferred embodiment illustrated in FIGS. 12 and 13, two tubes 124 are formed in the shaft 114. The body 120 has also been pre-bored to receive one or more screws 138 for attachment to the bone of the femur. As shown in FIG. 2, the screws may have expandable ends 188 for a firmer attachment to the bone, which is particularly desirable if the bone is soft.

A groove 140 is formed in the side wall 137 adjacent the bottom 128 to receive a U-shaped shield 142 that has a pair of legs 144 that extend outwardly from the groove. This shield is curved to fit the body 120 so that the legs will engage the corner of the cortical bone along the superior portion of the hole through the femur to reduce the risk that the bone will fail due to stresses applied by the body 120 of the shaft 114.

As discussed previously, when the patient's bone structure is large enough, a second preferred embodiment, prosthetic components 310, should be used, as illustrated in FIGS. 14, 22 and 23. In preferred embodiment 310, it was possible to insert thicker segments 420a-j (not all of which are shown) than those shown in FIG. 4, eliminating the need for inserting a second shell. Therefore the socket implant 312 comprises the segments 420a-j that are joined to one another to form the first shell 316 and a cup 406. The cup 406 is sized and configured to be received into the interior cavity of the first shell 416. The cup 406 is held in the first shell 416 by the snap ring 445. The cup 406 may be constructed from metal or from synthetic resin. Attachment by a snap ring may be done whether the cup 406 is metal or synthetic resin. The socket implant 312, is now completed. The shaft 414 is very similar in size and shape as shaft 114, described above, and the cup 406 will be sized to receive the ball 410 therein, which is generally the same size as ball 110.

The third preferred embodiment, prosthetic components 610, illustrated in FIGS. 15 and 27 are very similar in structure to the second preferred embodiment, prosthetic components 310, except a solid second shell has been inserted between the first segmented shell 616 and the cup 706. As shown in FIG. 15, the second shell 670 is formed as a single piece, that is, not segmented, as previously defined. The single piece shell 670 would have the same general shape as the segmented shell 70. The only limitation is that the diameter of the shell 670, at its maximum diameter, must be less than the diameter of the sleeve (not shown), which will be discussed further below. The second shell 670 is preferably made from metal, titanium or cobalt chrome. A synthetic resin cup 706 is inserted in the second shell 670 and attached by snap rings or heat and pressure as previously discussed. In this third preferred embodiment, the shaft 714 is very similar in structure to the shaft 114, and the cup 706 is sized to receive the shaft 714.

Having thus set forth a preferred construction for the current invention, is to the remembered that this is but a preferred embodiment. Attention is now invited to the use and the method for placement of the prosthetic implant of this invention.

These prosthetic components are used for surgical implantation when the patient is able to withstand a longer recovery period than that typically required by prior art methods. Recovery, in this case, may require the patient to be on crutches for a number of months to ensure complete healing before placing stress on the joint. An advantage of this apparatus and method is that the surgery is much less invasive, takes much less time, requires much less surgical support in the operating room, and is much less expensive. In addition, if these components were to fail, a full hip joint replacement, done in accordance with current practice, is still available, as more than enough of the femur remains and only a small portion of the acetabulum was removed. The steps for implantation of these prosthetic components are discussed below.

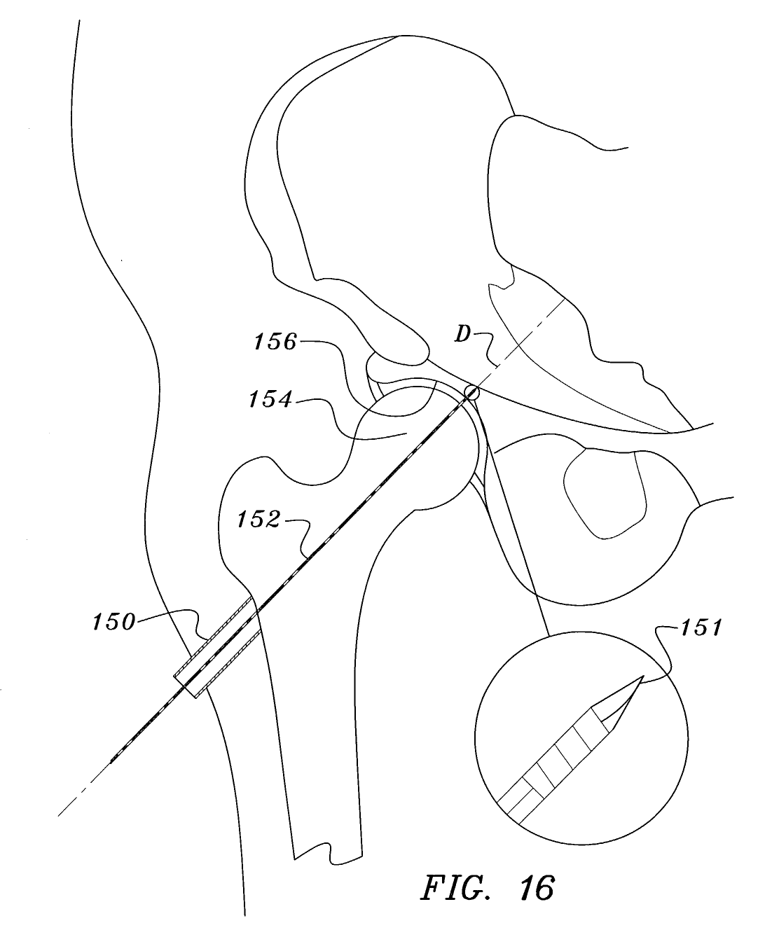

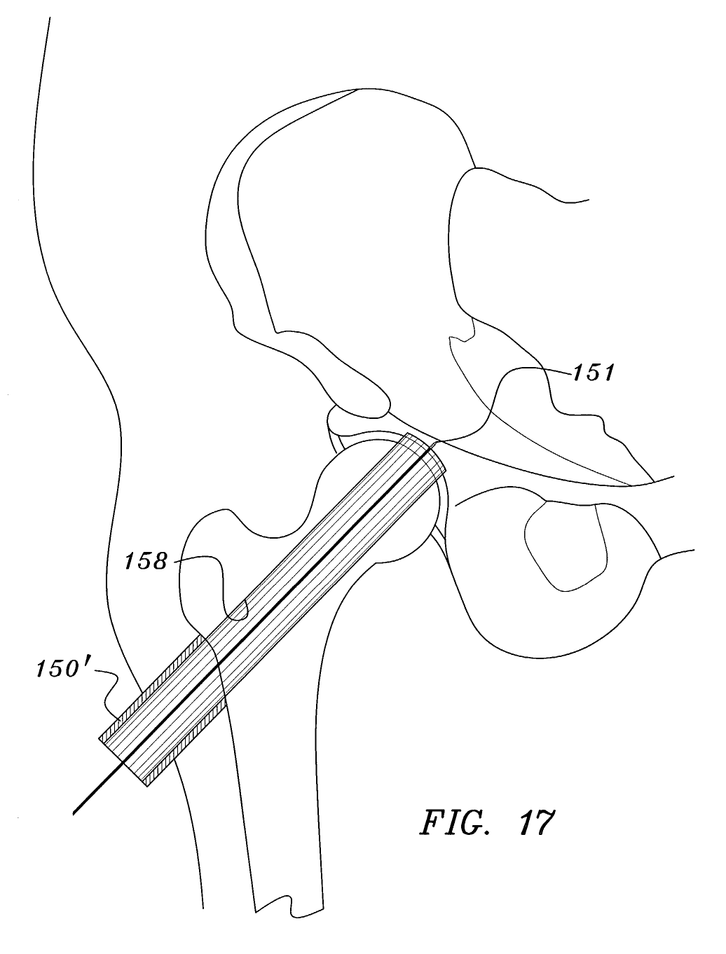

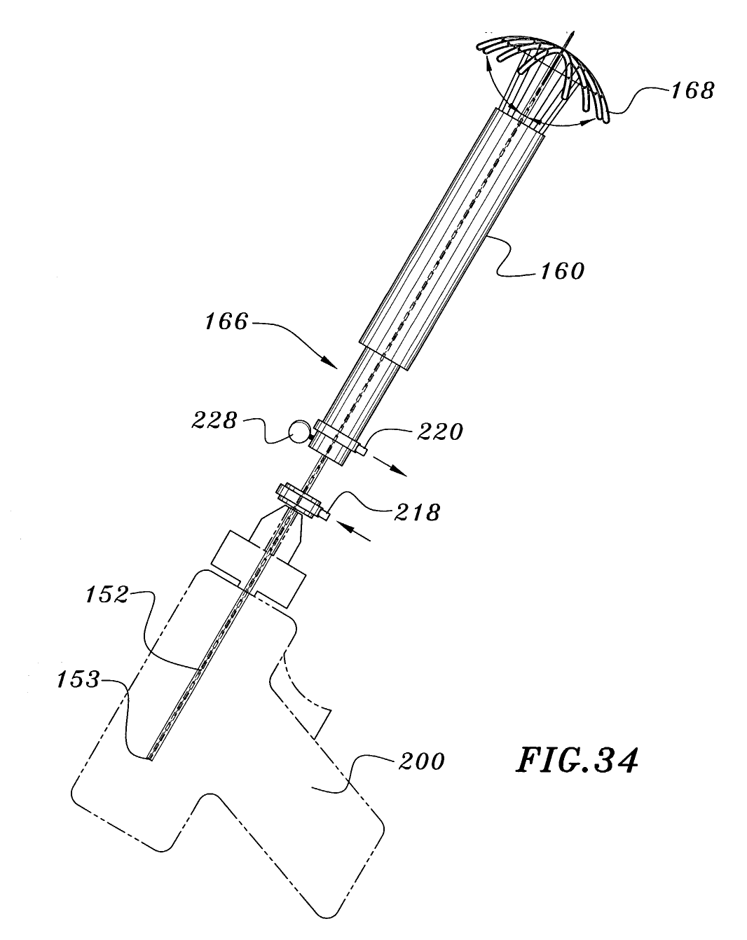

The patient is suitably prepared for surgery in accordance with known practice. The patient's body is aligned so that a longitudinal axis D extending from and neck and head of the femur of the patient passes through the geometric center of the acetabulum of the patient. The patient's body is placed in skin traction to prevent movement during surgery. A one inch incision is made to expose the femur at the point at which the longitudinal axis exits the femur, just under the greater trochanter. As seen in FIG. 16, a hollow and blunt guide 150 is inserted up against the bone so that its axis is coincident with the longitudinal axis D. A 2 mm smooth guide wire 152 with a threaded tip is passed through the guide 150 and along the longitudinal axis D so that the guide wire 152 is aligned with and driven through the geometric center of the femoral head, across the joint and into the geometric center of the acetabulum 156. A cannulated drill bit is mounted over the guide wire 152 and a hole is bored through the femur, the neck of the femur, the head, across the joint and 2 mm into the acetabulum. A larger blunt guide 150′ is placed over the smaller blunt guide 150, which is then removed. A second cannulated drill bit may be inserted in the drill to bring the hole 158 to the proper diameter, as shown in FIG. 17. The size of the hole 158 will largely be determined by the size of the femur neck. The hole 158 must not remove the interior of the subchondral plate. To ensure that the bore does not extend beyond the planned depth into the acetabulum, usually 2 mm, the cannulated drill bit and drill ride the guide wire 152 to a stop 153, as seen in FIG. 31. FIG. 34 demonstrates use of the drill motor 200 with the expandable drill bit 166; however a cannulated surgical drill bit may be inserted in the drill motor 200 and controlled in this manner. That is, the guide wire 152 is received within a tube in the drill motor 200 that has a predetermined length. The guide wire 152 is sized so that when the end of the guide wire 152 reaches the end of the tube, or stop 153, the proper length of the hole 158 has been reached.

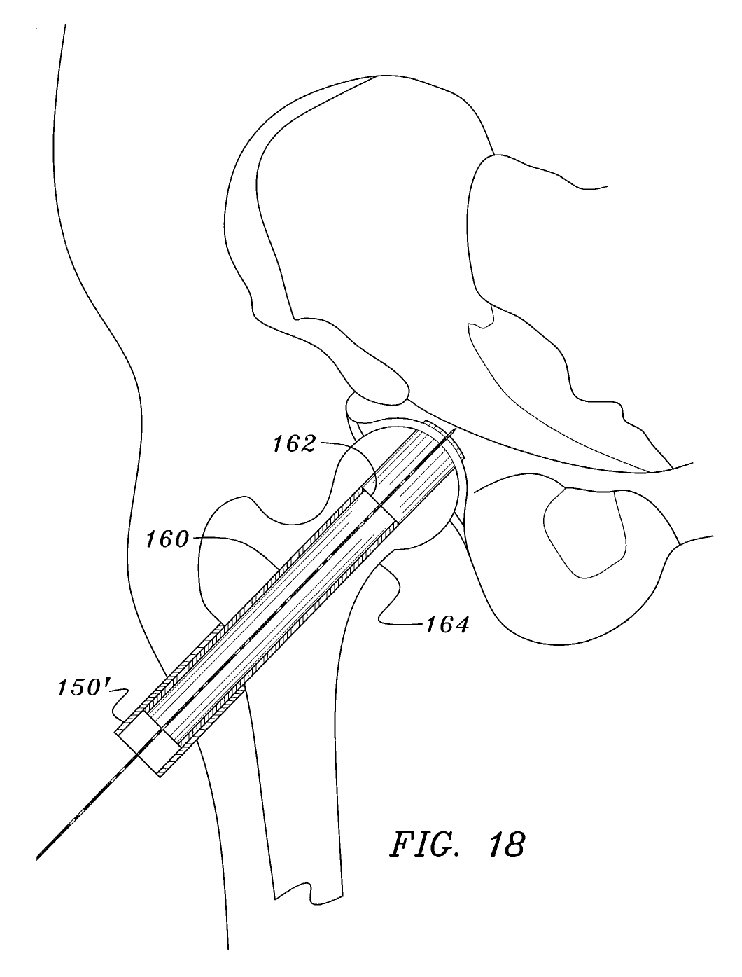

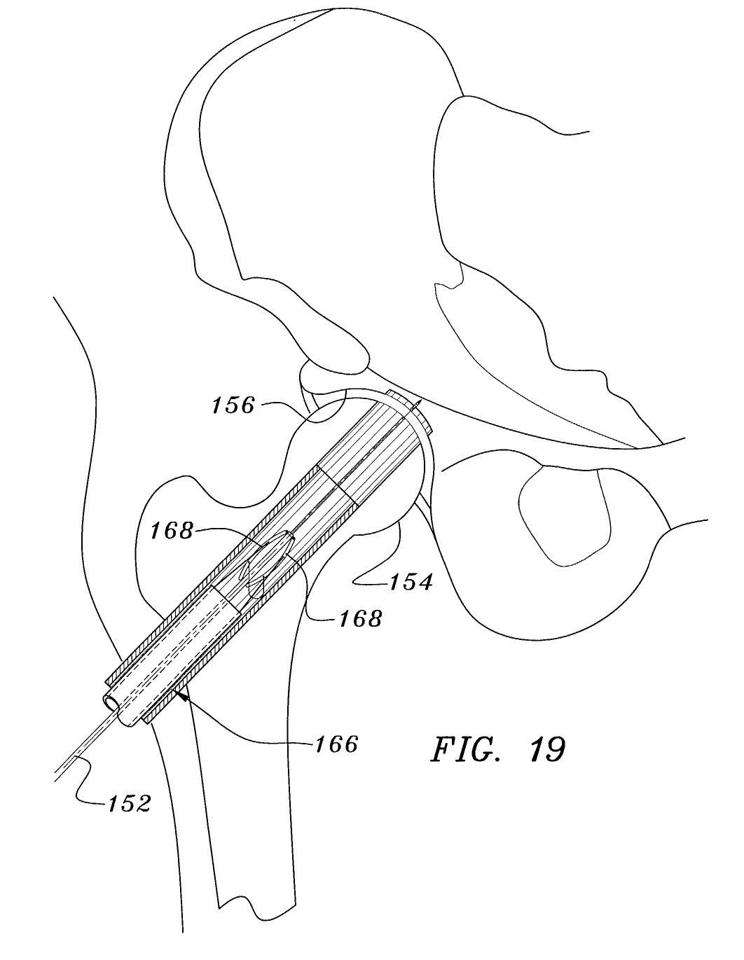

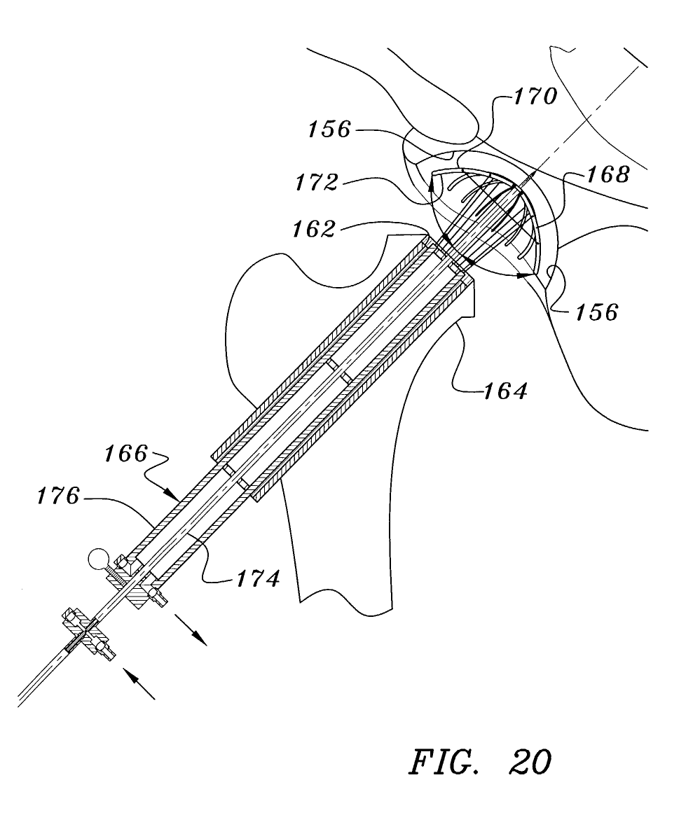

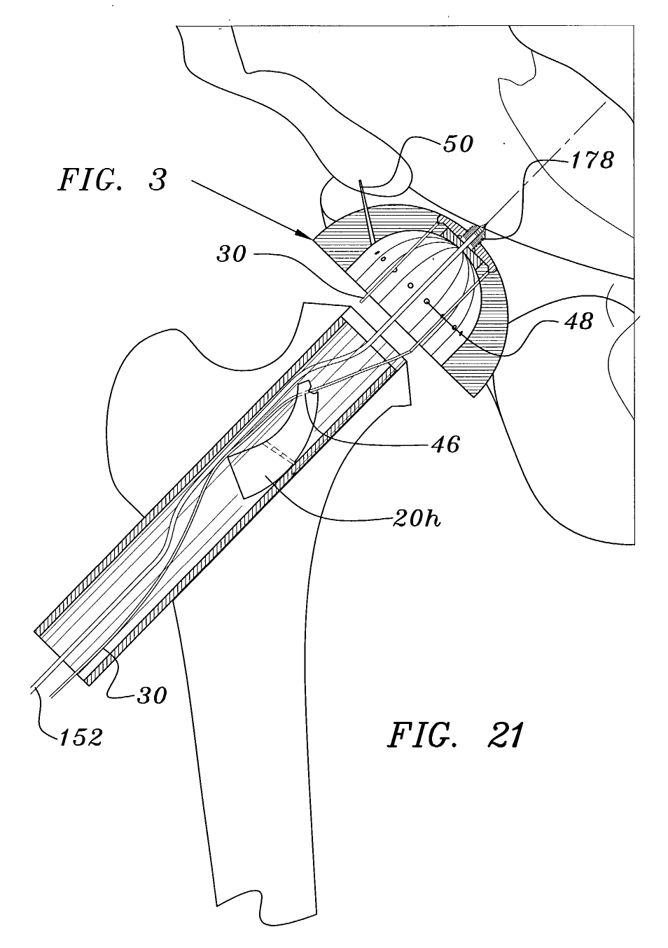

As seen in FIG. 18, sleeve 160, having the same exterior diameter of the hole 158 bored in the femur, is now placed around the wire and inserted through the blunt guide 150′ and into the femur until the first end 162 of the sleeve 160 lies proximal the neck 164. The blunt guide 150′ is then removed. An expandable drill bit 166, which will be discussed in greater detail below, is mounted on the guide wire 152 and inserted into the sleeve 160, as shown in FIG. 19. This drill has multiple blades 168 that can be expanded so that the outer cutting edges 170 of the blades 168, when fully expanded, match the spherical shape of the acetabulum. As seen in FIG. 20, once the blades have exited the sleeve 160 they begin cutting away the head of the femur 154. The expandable drill bit 166 is advanced until the drill blades 168 remove approximately 2 mm of the acetabulum 156. The expandable drill bit 166 includes means for measuring the distance that has been bored. Once the predetermined distance for movement of the drill along the guide wire has been reached the expandable drill bit 166 is drawn inwardly so that the inner cutting edge 172 of the blades 168 can remove additional portions of the head 154 of the femur that must be removed to ensure adequate clearance for full movement of the mechanical joint. The expandable drill bit 166 is advanced again until the blades 168 can be closed and then the expandable drill bit 166 is retracted through the sleeve 160. The drilling operation is rigidly controlled through fluoroscopy. While drilling is being accomplished a flushing fluid is injected into the site through a tube 174 in the expandable drill bit 166 and suction is applied to the central portion of expandable drill bit 176 for removal of debris.

Once the site has been cleaned of debris, a socket implant 12 is attached to the hipbone of the patient. The ball 110 is much smaller than the head of the femur, so that it can be inserted through the hole 158 through the femur, and the acetabulum site on the hipbone has been enlarged by the expandable drill bit 166. Therefore, the cup 106 receiving the ball must be much smaller, requiring a support structure between the hipbone and the cup 106 of the socket. The larger the cross-section of the neck of the femur the larger the hole 158 that can be bored through the femur, without damage to the subchondral plate, through which the socket implant is passed. The surgeon, based upon measurements of the patient's bone structure, will determine the particular size and structure of the socket implant. In the previous discussion of the preferred embodiments of the socket implant 12, three preferred embodiments of the prosthetic components, 10, 310 and 610 were discussed. A first preferred embodiment, prosthetic components 10, comprises a segmented first shell 16, a segmented second shell 70, and a solid synthetic resin or metal cup 106 and a shaft 114 as seen in FIG. 1. A second preferred embodiment, prosthetic components 310, comprise a socket implant 312 comprised of a metallic segmented first shell 316 and a metallic or synthetic resin cup 406, as seen in FIG. 14. A third preferred embodiment, prosthetic components 610, comprise the segmented first shell 616, a single piece metal second shell 670, and the cup 706, as seen in FIG. 15. Certainly, the less parts needed to complete the socket implant, the easier the placement and the more quickly the operation can proceed, which is better for the surgeon and the patient.

With the site prepared, and the surgeon having selected the first embodiment of the prosthetic components 10, the next step is to implant the segmented first shell 16. The base 18 having the cannulated screw 178 inserted therein, is mounted on the guide wire 152 so that the cannulated screw and base 18 passes along the guide wire 152 and is therefore centered in the acetabulum. With the base 18 centered by the guide wire the self-tapping cannulated screw 29 is driven into the bone of the acetabulum of the patient by a cannulated screw driver. The ends of the plurality of flexible spires 30 attached to the base 18 extend outwardly through and beyond the sleeve 160. The first segment to be implanted must be the segment having the two male arms 52, which in FIG. 5 is segment 20h. A flexible secondary guide wire 30 is received through the hole 46 in the first segment 20h so that the segment 28 is slid downwardly on the guide wire until the groove 44 on the first segment 20h engages the ridge 22 of the base 18, as seen in FIGS. 4 and 5. Once the segment is seated in place a surgical nail 50 is driven through the hole 48 in the segment 20h to attach the segment 20h to the hip bone. As seen in FIG. 19, some of the segments are already in place and one segment is shown mounted on one of the flexible secondary guide wires 30, so that the flexible secondary guide wire 30 passes through the hole 46 in the segment.

The next segment 20g is then mounted on its corresponding flexible secondary guide wire 30 and installed so that the female land 56 engages the groove 58 of the segment 20h. The next segments are inserted in the same manner until the next to last segment 20j is in place. The last segment, segment 20i, has two female arms 54 that engage with the male arms 52 on segments 20h and 20j interlocking the segments 20a-j of the segmented first shell 16 in place. In addition, as discussed previously, the segment 20i is tapered so that it may be inserted in place from the inside of the first shell 16. A surgical nail is driven through the hole 48 of segment 20i to lock the segments together. As seen in FIG. 21, surgical nails may be driven in more of the holes 48 in the segments 20a-j if the surgeon believes it is necessary. The location of the nails will be determined by the surgeon based upon the thickness and density of the bone in the adjacent area: however, they usually may be driven into the superior, posterior superior or the straight posterior portions of the hipbone. Certainly segments 20h and 20i should be located adjacent to bone that is thick and dense so that surgical nails 50 may be driven through these key segments. While FIG. 21 illustrates the method for placement of a segment, it also illustrates the completed implantation of the first segmented shell 16, with the plug 180 screwed into place, which, obviously could not happen until all the segments were in place.

As discussed previously, in another preferred embodiment a loop of surgical thread 64, as shown in FIG. 4A, may be used to guide the segments to their proper location. Certainly surgical thread is more flexible than wire and may create less difficulty in the installation of the segments.

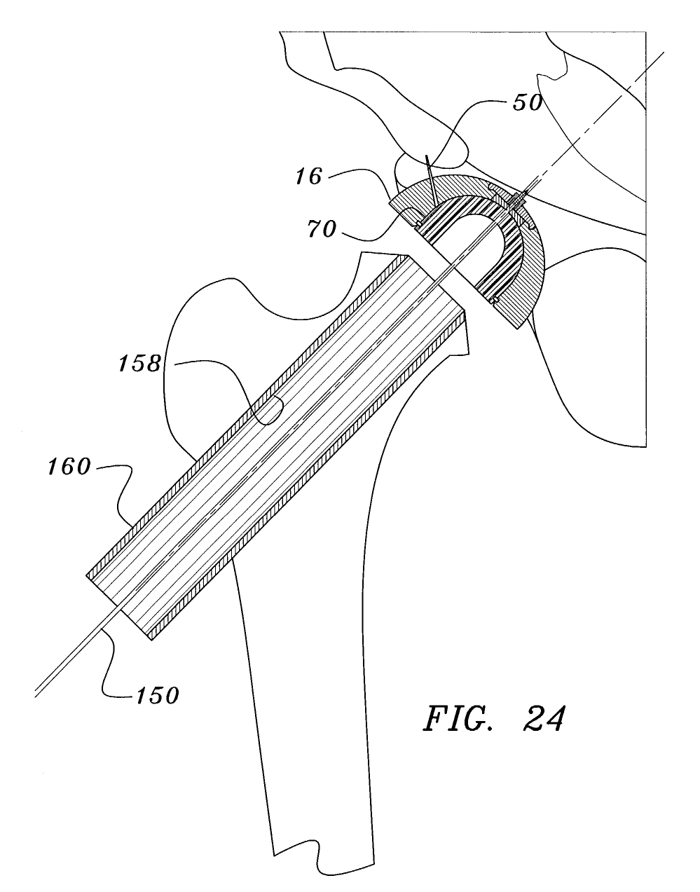

The surgeon has selected the prosthetic components 10 as the size of the femur is average or small, and the segments of the first shell 16 will need to be small enough to pass through the hole 158 in the femur. In this case, it will be necessary to place the segmented second shell 70 within the first shell 16 to fill the space formed in the acetabulum of the patient to support ball 110, which is much smaller than the head of the femur. FIG. 24 illustrates the implantation of such a preferred embodiment with a segmented second shell 70 having been inserted within the segmented first shell 16.

As shown in FIGS. 24-26, the second shell 70 lies between the first shell 16 and the cup 106 and is segmented. As shown in FIGS. 6-11, in a preferred embodiment, the segmented second shell 70 comprises a plurality of parts, a first group of parts 72a-e and a second group of parts 74a-e. The first group of parts 74a-e each have a hole 100 therethrough and the second group of parts each have a hole 102 therethrough. The parts are alternatingly strung on a surgical thread that is passed through the holes 100 and 102 and tied in a loop. The parts are then inserted through the sleeve 160 so that the guide wire 152 passes through the loop of surgical thread. The surgeon places the first group of parts within the first shell 16 beginning with part 72a and ending with part 72e. The last part 72e is tapered to provide a friction fit between part 72d and 72a, forcing the other parts into position forming a cup-shaped shell. The second group of parts 74a through 74e, which are held in proper orientation by the surgical thread, are then each inserted between the adjacent pair of the first group of parts 72a-72e. As seen in FIGS. 6 and 9, the second group of parts are tapered and may be readily placed in position and then firmly pushed into place. The parts 72a-e and 74a-e are then placed under pressure and high temperature (approximately 400 degrees), thereby bonding the plurality of parts 72a-e and 74a-e to one another and to the first shell 16.

When the second segmented shell 70 has the same configuration as the first segmented shell 16, it will be inserted through the sleeve 160 and assembled in the first segmented shell 16 in the same manner that the first segmented shell 16 was inserted through the sleeve 160 and assembled in the acetabulum. Of course, the first and last segments of the second shell will be preferably tacked to the first shell 16 to permit the attachment of the second shell to the first shell by heat and pressure or by a snap ring.

In other preferred embodiments, a layer of polymer cement may be applied in the place of the second shell 70. This cement will also seal the segmented shell and provide a means for attachment of the cup 106.

The next step is to insert a cup 106 through the sleeve 160 and attach it to the second shell 70 by a snap ring 188 that is inserted in the groove 190 in the parts 72a-e. As the cup is pushed into the interior cavity of the first shell 16, the exterior sides of the cup 106 engage the snap ring 188 pushing it into the groove 190 until the groove 190 aligns with the groove 182 in the cup 106, at which time the snap ring 188 expands outwardly and engages the groove 182 locking the cup 106 within the second shell 70, as seen in FIG. 25. The cup 106 may be constructed from metal or from synthetic resin. Attachment by a snap ring may be done whether the cup 106 is metal or synthetic resin; however, if the cup is made from synthetic resin it may be installed on the ball 110 prior to the shaft 114 being inserted in the hole 158, so that as the shaft 114 is pressed into the hole in the femur the ball 110 with the cup attached seats the cup 106 in the second shell 70 and is locked therein by a snap ring. As discussed previously the plastic cup 106 snaps on the ball as the edge 112 of the synthetic resin cup curves around the ball beyond its equator. The prosthetic acetabulum, or socket implant 12, is now completed.

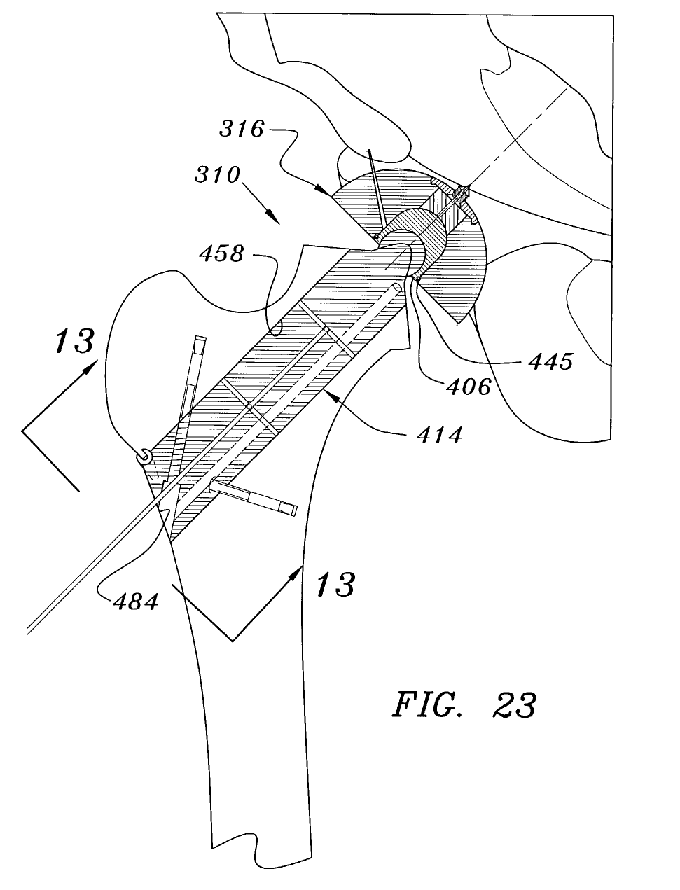

The sleeve 160 is then removed from the hole 158, and as shown in FIG. 23, the shaft 114 is implanted in the femur of the patient. The shaft 114 is driven into the hole 158 so that the ball 110 is seated within the cup 106 for free movement between the ball and the cup. As the shaft 114 is inserted, a shield 142 is placed around the superior portion of the hole 158 and is held in place by the groove 140 formed in the body 120. To stabilize the shaft 114 during the healing process, at least one surgical screw 138 is inserted through a hole 184 in the shaft 114. The surgical screw 138 has a first end 186 that is capable of expanding after it has entered into the femur to more tightly hold the shaft 114 in place. This is particularly necessary if the bones are soft. Additional screws may also be inserted through two other holes 184, as shown in FIG. 13. If the patient has very soft bones due to osteoporosis, a syringe is threadably attached to the tube 132 and a fluid cement is forced into the tube 132 and out the secondary tubes 136 and 136′ between the hipbone and the sidewall 137 of the body 120. Also, if the bones are soft a shaft with a larger diameter may be driven into the femur for a better bond, as long as the subchondral plate of the bone, the hard exterior layer of bone, is drilled to the size of the shaft.

The shaft 114 is cannulated by at least one tube 124 which permits flushing and suctioning of the hip joint site prior to closing the incision and at a later date, if infection or other difficulties occur. At this time, the surgeon may attach and implant a drainage bag constructed from a formulation of silicon and rubber, not shown, to catch any drainage after the incision is closed. The incision may now be closed. At a later date it may be necessary to remove and replace the drainage bag.

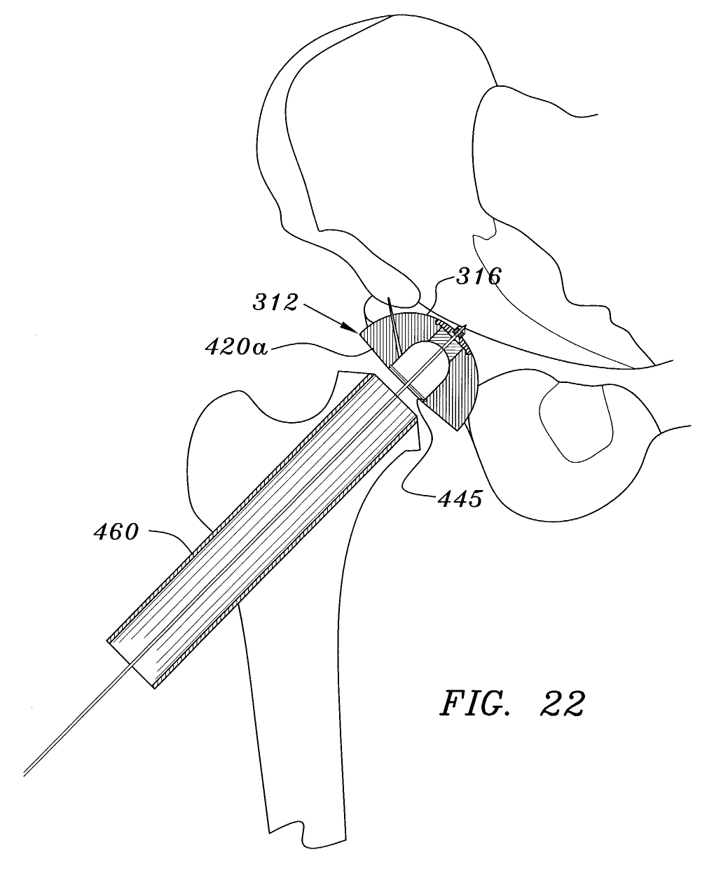

If the surgeon determines, through measurements of the patient's bone structure, that the bone structure can support the prosthetic component 310, the surgeon will normally select the prosthetic component 310 as it comprises fewer parts, thereby reducing the complexity of the operation. The steps for implantation of the prosthetic component 310 will largely be the same as the steps for implantation of prosthetic component 10, as described above. In this case, larger individual segments and possibly a greater number of segments will permit the much thicker segments of the first shell 316, as seen in FIG. 22, to be inserted through the sleeve 460. All the steps leading up to and for installing the first segment 316 are the same as described above. The thicker first shell 316 eliminates the need for installing a second segmented shell as contained in the prosthetic component 10.

The next step is to insert a cup 406 through the sleeve 460, the cup being sized and configured to be received into the interior cavity of the segmented first shell 316 and to be attached thereto. The steps for attachment of the cup 406 to the first shell 316, by snap ring 445 are the same as discussed above for attaching the cup 106 to the second shell 70.

The next steps relate to the installation of the shaft 414. The shaft 414 is installed by the same steps used to install the shaft 114, as described above. FIG. 23 shows the completed installation of the prosthetic component 310.

The surgeon may determine that the prosthetic component 310 will not make a proper fit without a second shell, but that the second shell could be formed as a single piece as shown in FIGS. 15 and 27 and still pass through the hole 458 as shown in FIG. 27.

Therefore, he would select the prosthetic component 610, as shown in FIG. 15. The steps for installation of the prosthetic component 610 through the step for installation of the enlarged segments, will be the same steps as the steps for installation of the prosthetic component 10 through the installation of the first shell 116. The steps for installing the single piece second shell 670 will require that the shell 670 be sized to pass through the hole 458. The shell 670 will then be inserted into the interior of the first shell 616 and attached thereto by a snap ring, in the same manner that the cup 106 was attached to the second shell 70, as discussed above. In a preferred embodiment of the prosthetic component 610, the solid (single piece) second shell 616, when made of synthetic resin, may be bonded to the first shell by pressure and temperature.

The next steps relate to the installation of the shaft 714. The shaft 714 is installed by the same steps used to install the shaft 114, as described above. FIG. 27 shows the completed installation of the prosthetic component 610, with the ball 710 inserted into the cup 706.

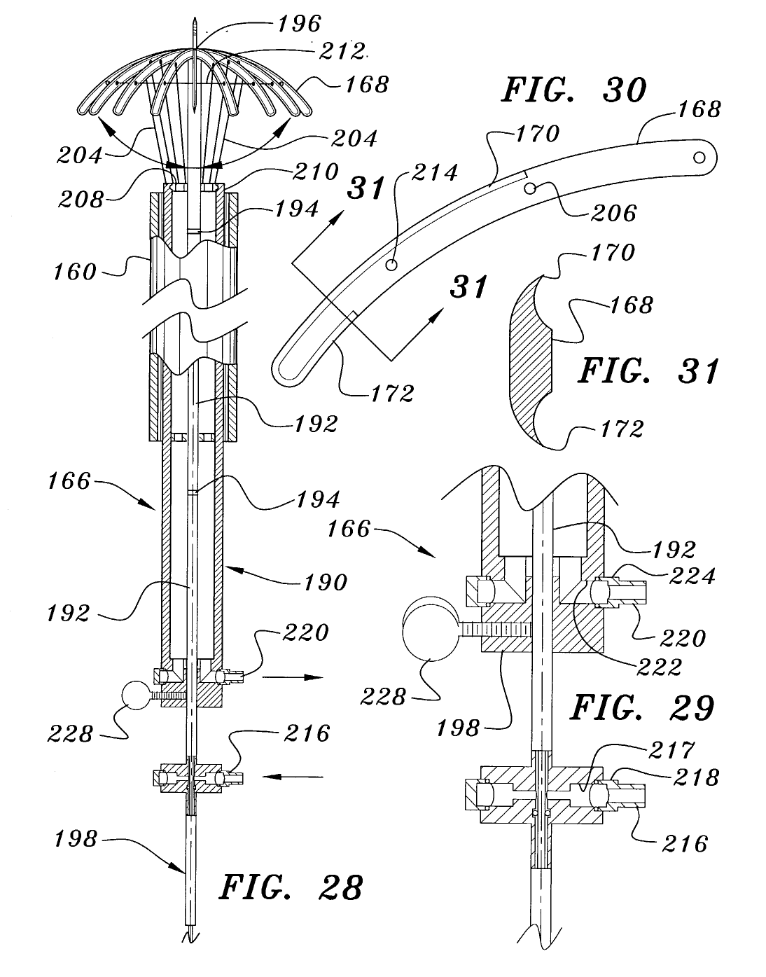

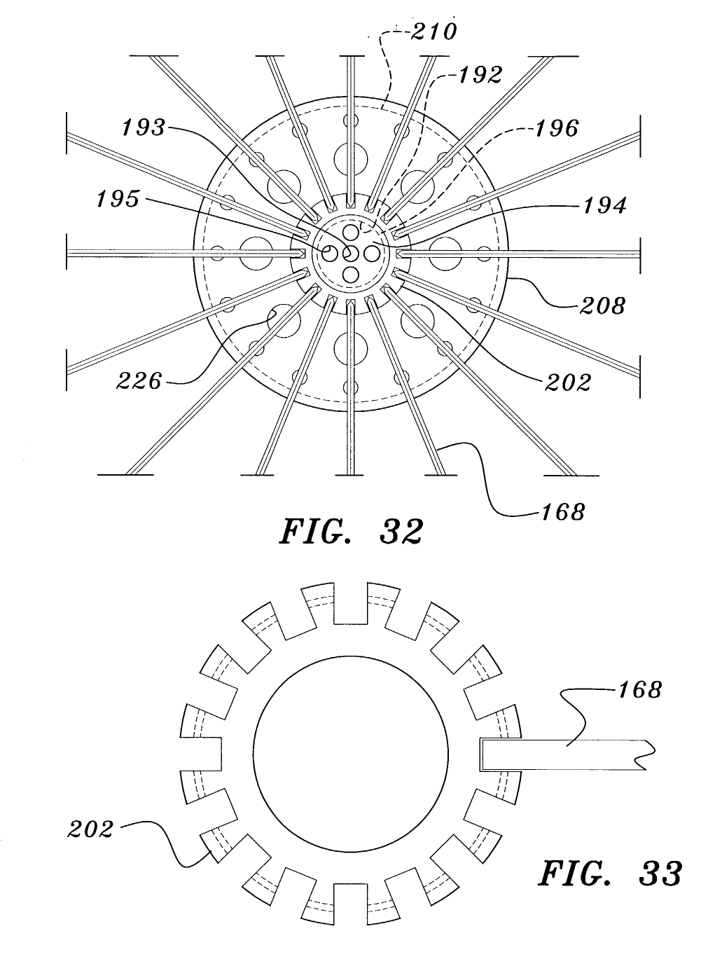

The steps for implantation include the use of an expandable drill bit 166 for removal of the head of the femur and a portion of the acetabulum prior to placement of the prosthetic components. FIGS. 28-33 disclose a preferred embodiment of an expandable drill bit 166. In FIG. 28, the expandable drill bit is shown extending through the sleeve 160. The expandable drill bit 166 comprises a plurality of blades 168, a body 190 and a central hollow shaft 192 that includes a plurality of supports 194. As seen in FIG. 29, the top plan view of the apparatus of FIG. 32, a support 194 is attached to the first end 196 of the shaft 192, and at least one additional support 194, as seen in FIG. 28, is spaced along the length of the shaft 192. The support 194 has a hole 193 therethrough for receiving the guide wire 152 therethrough and a plurality of holes 195 that permits water to pass therethrough for flushing the site. The second end 198 of the shaft 192 is insertable in a drill motor 200, as shown in FIG. 34, for rotation of the shaft 192 and the blades 168 of the expandable drill bit 166.

Annular plate 202, as shown in FIGS. 32 and 33, is attached to the first end 196 of the shaft 192 for attachment of the blades 168 to the shaft 192. Each blade 168 has one end of a stiff wire 204 attached to a hole 206 in a respective blade of the plurality of blades 168. The other end of the stiff wire 204 is attached to the end support plate 208 which is mounted to the first end 210 of the body 190. The body 190 is slideably mounted on the shaft 192 for expansion and retraction of the blades 168. A flexible wire 212 is passed through the holes 214 of the blades 168 as a safety measure to prevent the blades from expanding beyond a predetermined arc with a radius matching the finished radius of the acetabulum.

The hollow shaft 192 provides a means for delivering a flushing fluid to the cutting site through the port 216, which is connected to a pressurized water supply, (not shown). The port 216 is connected to a fixed annular ring 218, as seen in detail FIG. 29, that is sealingly attached to an annular cavity 217 that extends about the body 190 so that the body 190 may rotate inside the ring and maintain the port 216 in fluid flow communication with annular cavity 217 and the hollow shaft 192. A water source is attached to the port 216 by any well known means. Suction may be applied to port 220 by any well known suction device (not shown). Port 220 is connected in fluid flow communication with the interior of the body 190 through a fixed annular ring 224, for rotation of the body 190 therein, and an annular cavity 222. This permits suctioning the flush water and debris from the hip joint site through the plurality of holds 226 through the end plate 208 and the hollow body 190.

As the body 190 is free to slide longitudinally on the shaft 192, it is also free to rotate about the longitudinal axis of the shaft 192. During a cutting operation the body 190 must rotate with the shaft 192 to maintain the wires 204 in proper orientation. Therefore, thumbscrew 228 is tightened to rotate the body 190 with the shaft 192 and is loosened when adjustments are made to the angle of the blades 168. For adjustments to be made to the blades 168, the drilling must be stopped, the thumb screw 228 loosened, and the body moved along the shaft 192.

As discussed previously, each blade 168 has an outer cutting edge 170 and an inner cutting edge 172. The outer cutting edge is used primarily for cutting through the head of the femur and cutting the acetabulum to its predetermined curvature. The inner cutting edge is used to further trim the neck and head of the femur to ensure adequate clearance for free movement of the prosthetic joint.

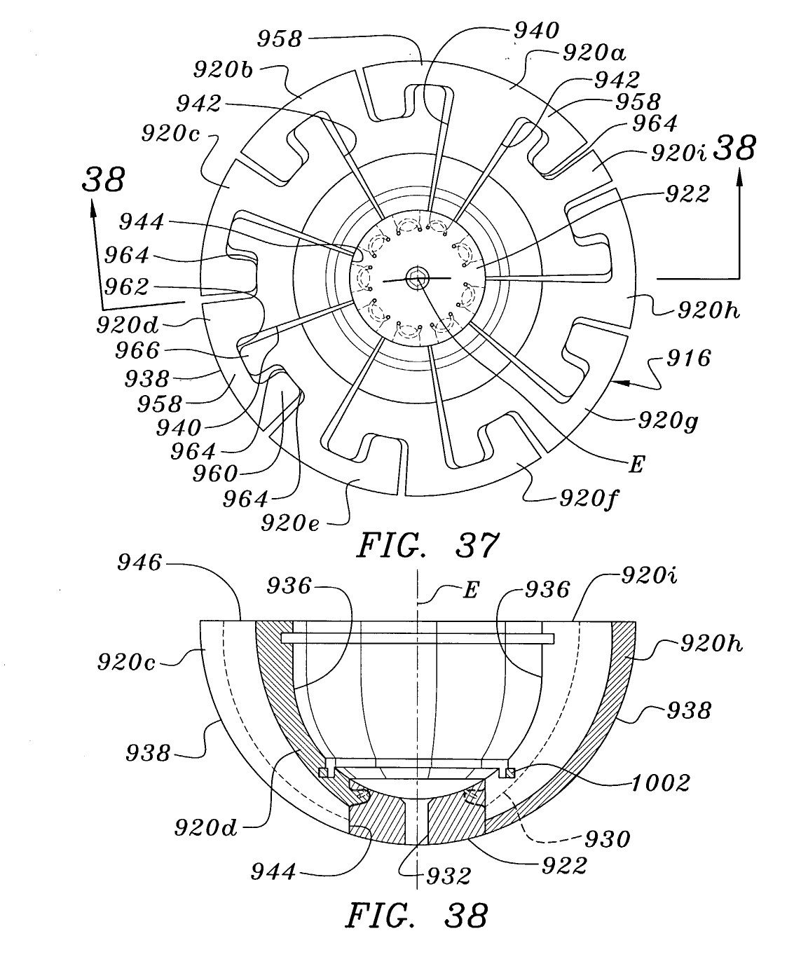

A fourth preferred embodiment for the prosthetic components of this invention are illustrated in the drawing FIGS. 35-53, in which the apparatus is generally indicated as 910. As mentioned previously, reference characters increased by 900 for these drawings will not necessarily refer to similar elements in the first embodiment, the specific drawings FIGS. 35-52 must be referred to. The prosthetic components comprise a socket implant 912 and a ball implant, conveniently shaft 914. A preferred embodiment of the socket implant 912 is illustrated in FIGS. 37-43, and comprises a segmented shell 916 made from titanium or cobalt chrome and a cup 918 made from titanium or cobalt chrome, synthetic resin or ceramic materials. In other preferred embodiments, structure of the socket implant 912 of the prosthetic components 910 will be modified according to the need, to form an additional preferred embodiment of the socket implant 1012.

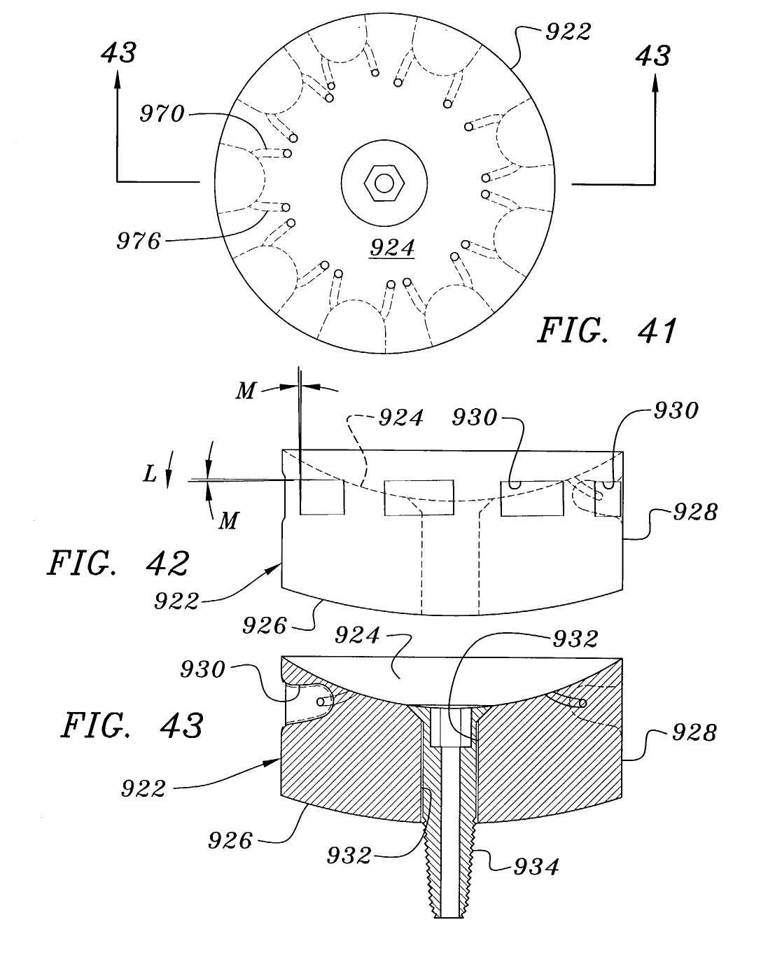

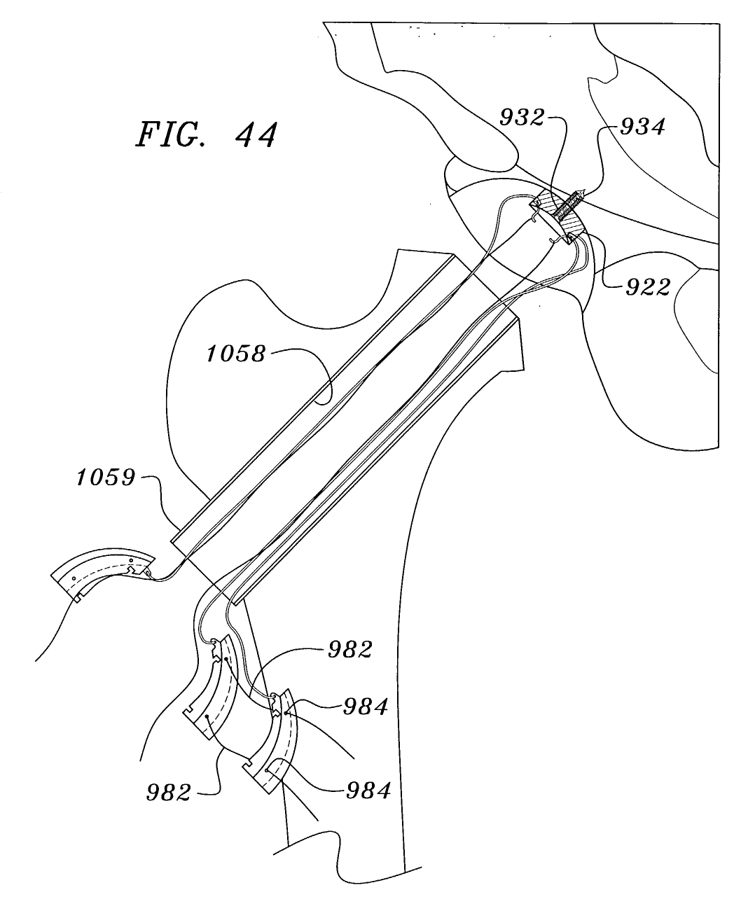

The segmented shell 916, which will be mounted into the prepared hip acetabulum is comprised of a plurality of segments 920a-i that engage one another and a base 922. The term segment is defined as a separate piece of something and segmented is defined as something that is comprised of separate pieces. Any convenient number of segments can be used; however, nine segments have been used for illustrative purposes. Therefore the segmented shell 916 is comprised of separate pieces or segments 920a-i that are joined together with the base 922 to form the segmented shell 916. As seen more clearly in FIGS. 41-43, the base 922 is comprised of a top surface 924, a bottom surface 926 and at least one side 928. The base 922 has a plurality of niches 930 formed therein that extend into the base 922 through the side 928. The base 922 has a hole 932 therethrough for receipt of a cannulated screw 934 for attachment of the base 922 to the hip bone, as seen in FIG. 44. The bottom surface 926 of the base 922 is curved to fit the curvature of the prepared surface on the hip bone.

As seen in FIGS. 37-40, each segment 920a-i has a pair of opposing faces, inner face 936 and an outer face 938, a pair of opposing sides, a longitudinally extending first side 940 and a longitudinally extending second side 942, a first end 944 and a second end 946. The faces 936 and 938 and the sides 940 and 942 extend between the first end 944 and the second end 946. The outer face 938 of each segment 920a-i is longitudinally and transversely arcuate, with a curvature that will match the curvature of the prepared surface of the hip bone. At least one of the segments has a hole 948 that extends through the inner and outer faces 936 and 938 of the segment intermediate the first and second ends 940 and 942 of the segment. One or more of the holes 948 may receive a surgical nail 950 therethrough, particularly the first segment 920a, if desired by the surgeon to attach one or more of the segments 920a-i to the prepared bone surface. A nail 950 being visible in FIG. 936.

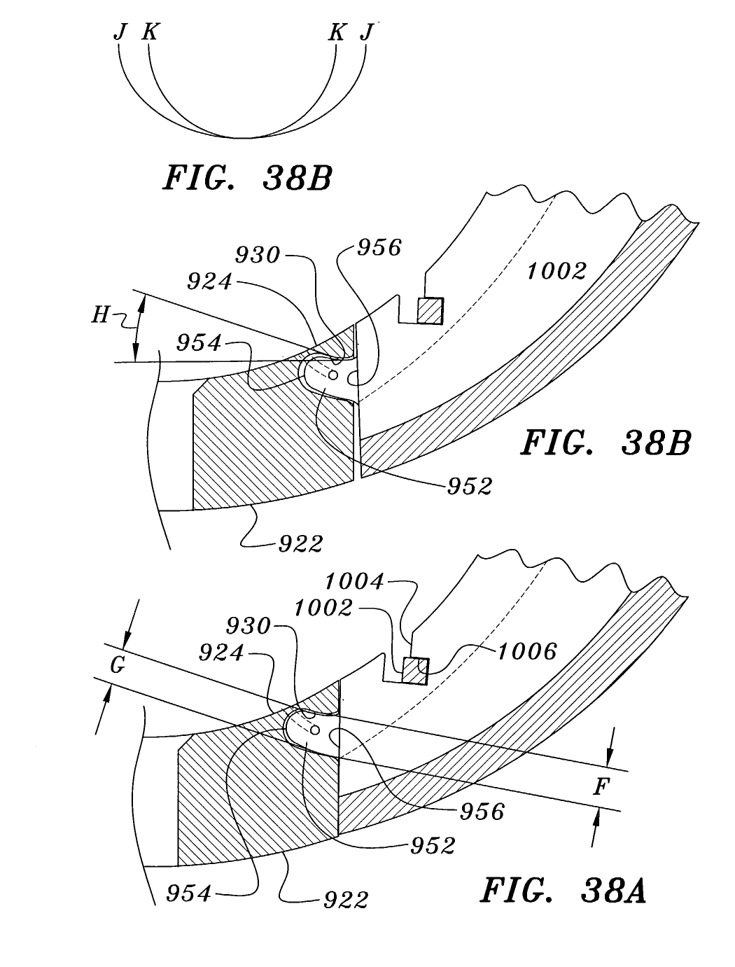

Each segment 920a-i has a projection formed on and extending outwardly from the first end 944 of each segment 920a-i, which is defined as a tooth 952. Each tooth 952 has a first end 954 and a second end 956, which is attached to the first end 944 of the segment. As seen in FIG. 4B, each tooth 952 is tapered from the first end 954 toward the second end 956 so that the thickness F is greater than the thickness G. In addition, as seen in FIG. 38A a portion of the tooth 952 that is proximal to the first end 954 is angled toward the top surface 924 of the base 922 at an angle H. The niche 930 is designed and configured to match the taper and the angle formed in the tooth 952, when the tooth 952 is fully inserted into the niche 930 as seen in FIG. 38B. Therefore, each niche 930 is also tapered and a portion of each niche that is spaced apart from the open-end of each niche 930 is angled toward the top surface 924 of the base 922.

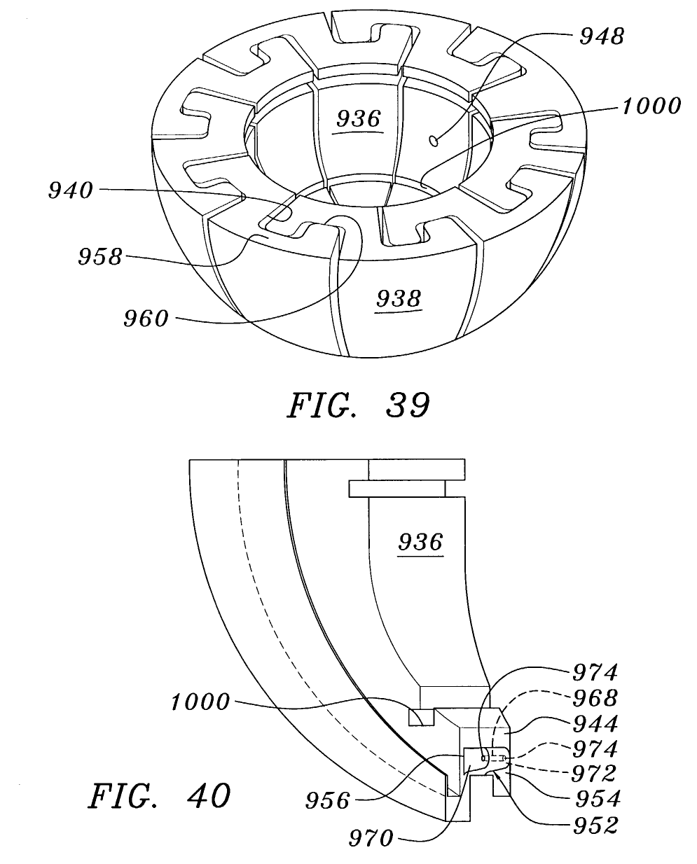

As seen most clearly in FIGS. 37 and 39, all but two segments, segments 920a and 920i, of the segments 920a-i have the same interlocking structure. The segments 920b-h are identical, each having a leg 958 that extends outwardly from the first side 940. The first end 960 of each leg 958 extends inwardly toward the axis E and of the shell 916. The angled end 960 creates a channel 962 that lies between the first side 940 of each segment 20a-28h and the first end 960. The outer face of each segment 920b-920h has a slot 964 formed therein. The slot 964 and the second side 942 of each segment 920b-920h form an outwardly extending ridge 966 that has been radially shortened so that it may be received within the channel 962 of the adjacent segment. Therefore, adjacent pairs of segments, for example segment 920d and segment 920e, engage one another when the first end 960 of the first segment, 920d, of the pair of segments, is received in the slot 964 of the second segment 920e, of the pair of segments, which then causes the ridge 966 of the second segment to be received in the channel 962 of the first segment. It is noted that the ridge 966 is narrower than the channel 962 and that the first end 960 of the leg is narrower than the slot 964 to permit movement between the adjacent segments, which will be discussed further below.

Segments 920a and 920i identify respectively a first segment and a last segment. These two segments are structured differently so that the shell of segments may be closed. The first segment 920a has a leg 958 extending from the first side 940 and a leg 958 extending from the second side 942. The last segment 920i has two slots 964 formed in the outer surface 938, which permits the ridges 966 of the last segment 920i to be received by the corresponding channels 962 of the adjacent segments 920a and 920h.

In a preferred embodiment, segment 920i is preferably wedge shaped, with the arc of the outer surface 938 being narrower than the arc of the inner surface 936. The adjacent sides of the two adjoining segments, 920a and 920h, are also modified to receive the wedge shape of the segment 920i.

In a preferred embodiment of prosthetic components 910, the shell 916 is constructed from titanium or cobalt chrome. In other preferred embodiments of prosthetic components 910, other materials that are very resistant to wear are suitable for the purpose.

In a preferred embodiment of prosthetic components 910, the shell 916 will be received by the surgeon in disassembled form for thorough disinfecting prior to assembly in the patient by the surgeon. To assist in the assembly of the shell 16, each tooth 952 of each segment 920a-920h has a hole 968 passing through the opposing sides 970 and 972 of the tooth 952, creating an opening 974 in opposing sides 970 and 972 of the tooth 952. As seen in FIG. 41, each niche 930 has a pair of conduits 976 extending from each niche 930 through the top surface 924 of the base 922, so that when the tooth 952 is inserted within the niche 930 each opening 974 is aligned with one of the conduits of the pair of conduits 976 as seen in FIG. 38. The use of the holes and conduits will be discussed further below during discussion of the method for installation of this preferred embodiment of the joint prosthesis 10.

As seen in FIG. 42, in a preferred embodiment the niche is 930 may be rotated in the direction of the arrow L during manufacture of the base 922 through a very small angle M, which will cause the ridge 966 to tighten against the channel 962 of the previously installed segment creating a tighter fit between the segments.

As seen in FIG. 44, the patient has been prepared for implantation of the hip joint prostheses 910 in accordance with the method previously described in the first embodiment. As seen in FIG. 44 the base 922 has already been implanted in the hip bone with a cannulated screw 934. This was done by using the same steps in which the base 180 was attached to the hip bone in the first embodiment. That is, by passing the base 922 through the bore 1058 through the femur which has been lined with a sleeve 1059. The base 922 is guided by mounting it upon a guide wire that has been embedded in the center of the patient's acetabulum. A cannulated screw 934 was then passed down the same guide wire and through the hole 932 of the base 922. The cannulated screw 934 is then screwed into the bone to firmly hold the base to the hip bone.

As seen in FIG. 44, a pair of segments are shown mounted on loops 978 of very flexible 1.3 mm woven stainless steel wire with a 100 pound pole strength. Each closed loop 978 passes through one conduit through the hole 968 in the tooth 952 of one of the segments and then out through the other conduit of the pair of conduits. All the segments are arranged in this fashion prior to the attachment of the base 922 to the hip bone. The segments are arranged outside the sleeve 1059 until the base is attached to the hip bone. A thread 980 may be attached to the loop as it spans between the pair of conduits across the top surface 924 of the base 922. In this way the loop can be formed of a fixed size, so that all loops are identical and the thread may be pulled to bring the end of the loop proximal the base to a point outside the sleeve 1059, which at the same time causes the segment to pass through the sleeve 1059 and the tooth 952 to enter the corresponding niche 930. Once the tooth of each segment has entered the corresponding niche, the next segment is installed. It may be necessary to assist in the placement of the segments through the use of a long tool that can be inserted through the sleeve and used to adjust the alignment of any errant segment.

In other preferred embodiments, the method for implanting may include guide threads 982 that are attached to the first segment to be installed. The guide threads 982 are passed through holes 984 in each additional segment, so that the segments may be guided into proper alignment with the adjacent segment already installed. As soon as all the segments are loosely aligned with one another these guide threads are removed.

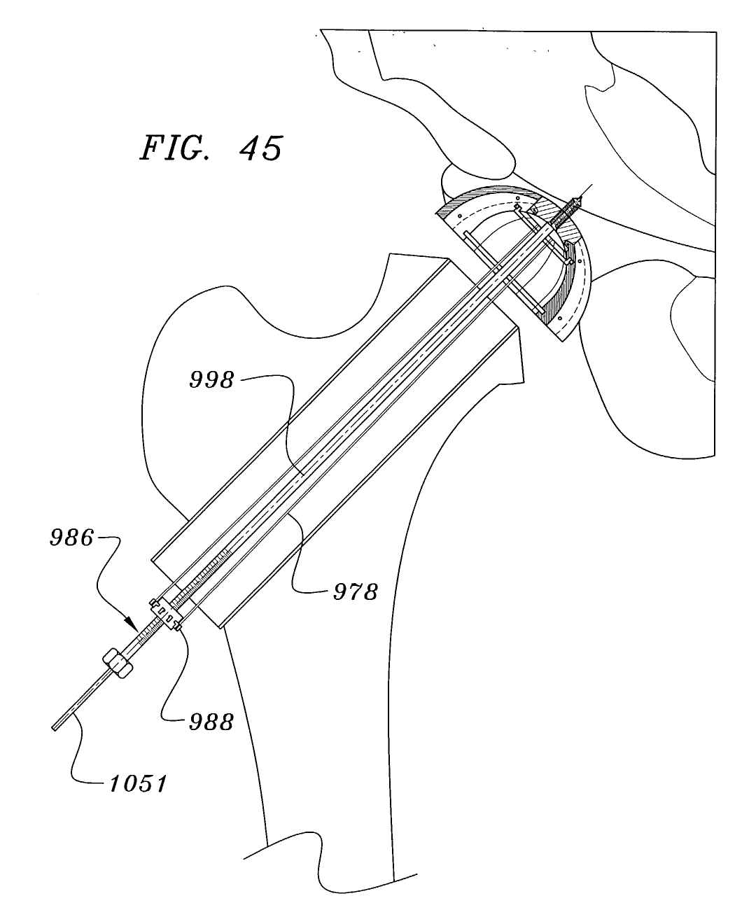

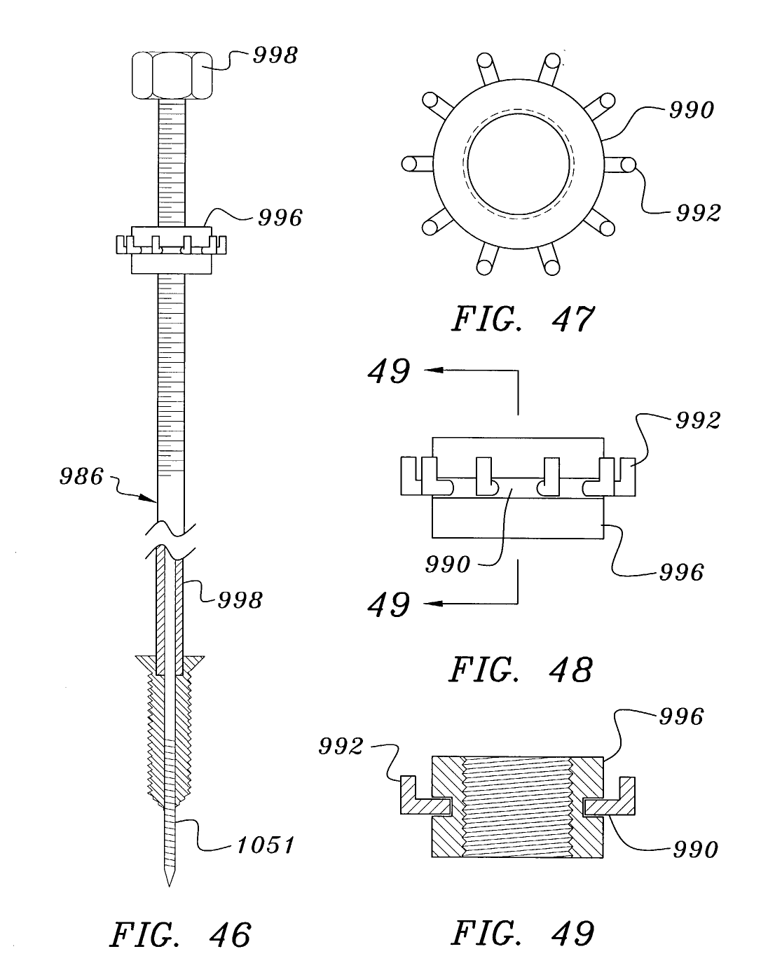

As seen in FIG. 45, a tensioning tool, conveniently a tensioner 986 is used to equally tighten each segment's tooth 952 into its corresponding niche 930. The tensioner 986, as seen in FIGS. 46, 47, 48, and 49, comprises a hollow tube 988 that is sized to receive the guide wire 1151 therethrough. When placed over the guide wire the tube rests on the cannulated screw and is rotatable thereagainst. A ring 990, having a plurality of hooks 992 thereon, is mounted in an annular slit 994 in a threaded sleeve 996. A portion of the tube 998 is threaded for receipt of the threaded sleeve 996, as seen in FIG. 46. A nut 998 is fixedly attached to the end of the tube 988.

Each loop 978 is placed over a corresponding hook 988 on the tensioner 996. The tube 988 is rotated until tension is applied to the loops 978. A torque wrench may then be applied to the nut 998 and the tube 988 rotated until a predetermined torque is reached and a predetermined tension is applied to the wire loops 978, firmly inserting the teeth 952 into the corresponding niches 930. The loops 978 are then cut and removed.

An L-shaped annular lock ring recess 1000 is formed in the shell 916 with a portion of the lock ring recess 1000 extending across the interior surface 936 of each segment 920a-i, as seen in FIGS. 39 and 40. A lock ring 1002 is inserted into the lock ring recess 1000 after tension is applied to the wire loops 978. The lock ring 1002 can be seen in FIGS. 38, 38A, and 38B. Before the lock ring 1002 is inserted in the lock ring recess 1000, it is cooled by liquid nitrogen to between −100 and −150 degrees Fahrenheit causing the ring to contract. The stem portion 1004 of the lock ring recess is sized to receive the cooled lock ring. As the lock ring warms it expands outwardly into the base 1006 of the lock ring recess and pushes against the segments locking them in place.

The segments 920a-i of the segmented shell 916 are circumferentially loosely attached to one another as described above, and as seen in FIGS. 37 and 39. This permits a particularly sized segmented shell to be used in acetabulums whose radius is within a specific range of radii. As seen in FIG. 38B, when the teeth 952 are fully inserted within the corresponding niches 930 each segment is pivoted outwardly so that the segmented shell 916 forms a shell with the maximum radius and maximum circumference J, as seen in representation FIG. 38C, for that shell. This occurs because a portion of the teeth and the niches are angled upwardly, as described above, so that as the tooth enters the niche it pivots outwardly. As seen in FIG. 38A, when the tooth is only partially inserted within its niche the shell has the minimum radius and minimum circumference K, as seen in the representation of FIG. 38C. With each sized segmented shell fitting a range of acetabulums, it permits stocking fewer different sized prosthetic segmented shells 916.

Once the segmented shell 916 is implanted in the hip bone, the shaft 914 may be installed in the bore 1058 of the femur. The shaft 914 is the same as the structure of shaft 114 as shown in FIGS. 12, 13 and 14, with but a few differences.

The ball implant, conveniently shaft 914, as shown in FIGS. 35 and 36, has a first end 1008 and a second end 1010. The shaft 914 is comprised of the ball 1012, a body 1014, and a neck 1016 that attaches the ball 1012 to the body 1014. This can be clearly seen in FIG. 36, 50 and FIG. 51. In this preferred embodiment, the ball and the neck and the body are formed from one piece of material made of titanium or cobalt chrome, which is cut to the required shape and size. The ball 1012 is generally spherical, is highly polished and is sized to be received in the cup 918. The body 1014 is coated with any one of the well-known ingrowth surfaces for encouraging the growth of the bone to the surface of the body, thereby bonding the body 1014 to the adjacent bone of the femur. The shaft 918 is cannulated, having at least one tube 1018 that extends from an open first end 1020 at the bottom 1022 of the shaft 918 to an open second end 1024 at the neck 1016. The tube 1018 is used to flush and suction the site of the hip joint to remove debris that may have collected in the joint area due to the wear of the ball 1012 or cup 918 and may also be used to insert antibiotics to fight infection that may occur after implantation of the prosthetic components 910. In a preferred embodiment as seen in FIGS. 36, 50 and 51, an annular skirt 1026 having a first end 1028 that is sealingly connected to the neck 1016 and a second end 1030 that is sealingly connected to the cup 918, by well known means, providing a sealed area to collect any debris created by the motion of the ball 1012 within the cup 918. The first end 1028 is so attached to the neck 1016 that the open second end 1020 of the tube 1018 lies within the sealed area created by the skirt 1026. A plug 1032 is inserted within the tube 1018 proximal to the first open end 1020 of the tube 1018. The plug 1032 is constructed of a self ceilinged material that is penetrable by hollow needle so that the tube and joint area may be flushed and suctioned to remove debris from the sealed area created by the skirt 1026.

An open second tube 1034 also extends inwardly from the bottom 1022 of the shaft 914 to a closed second end 1036 proximal the neck 1016. At least one secondary tube 1040 is connected at one point along the second tube 1034 and is in fluid flow communication therewith. In a preferred embodiment there are a plurality of secondary tubes 1040. The secondary tube 1040 extends through the side wall 1042 of the body 1014 so that a polymer cement may be applied between the body 1014 and the surrounding bone. For an even distribution of the cement around the body 120, a plurality of secondary tubes 1040 interconnect with the second tube 1034. The second tube 1034 has an open first end 1038 that is threaded for attachment of a syringe through which the cement may be injected into the second tube 1034 and into the secondary tubes 1040. The threads on the second tube 1034 may also be used for attachment of an extraction tool (not shown) to remove the shaft 914, if that should be necessary.

The body 120 has also been pre-bored to receive one or more screws 1044 for attachment to the bone of the femur. As shown in FIG. 36, the screws may have expandable ends 1046 for a firmer attachment to the bone, which is particularly desirable if the bone is soft. This preferred embodiment also has the U-shaped shield 1048 to reduce the risk that the bone will fail due to stresses applied by the body 1014 of the shaft 914.

Just prior to the implantation of the shaft 914, with the cup 918 attached, the surgeon may apply a well-known biologically tolerable bonding resin or polymer cement to seal the face of the segmented shell 916, and to bond the cup 918 to the segmented shell 916. The cement layer may be sufficiently thick that it can replace an intermediate solid or segmented layer, which in previous embodiments was inserted between the first segmented shell and the cup. In addition, the cup may be attached to the segmented shell 916 in a different way, which may be used alone or in combination with the cement. Snap rings or drop rings operate simply. Snap ring 1050, as seen in FIG. 51, is inserted within an annular groove 1052 in the shell 916, that matches a second groove or shoulder 1054 in the cup 918. When the cup 918 is pressed into place, the snap ring 1050 expands into the groove 1052 so that when the groove or shoulder 1054 aligns with the groove 1052 the snap ring shrinks into the groove or beyond shoulder 1054 locking the cup 918 into the shell 916.

Once the cup 918 has been received by the shell 916, the body 1014 is attached by the screws 1044 to the bone of the femur. The incision may then be closed.

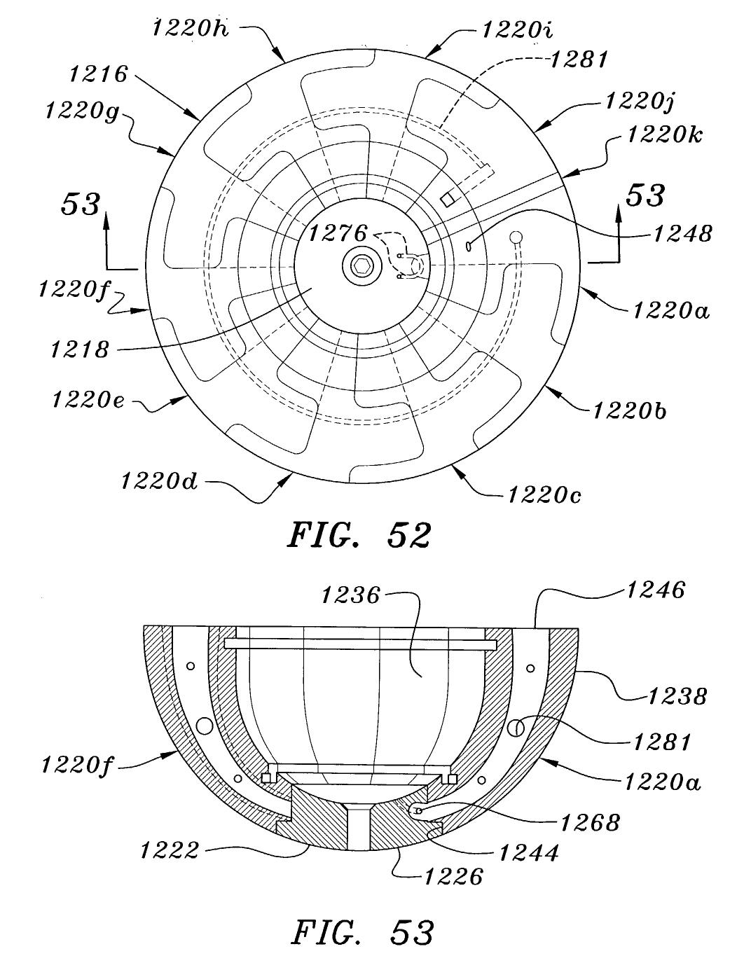

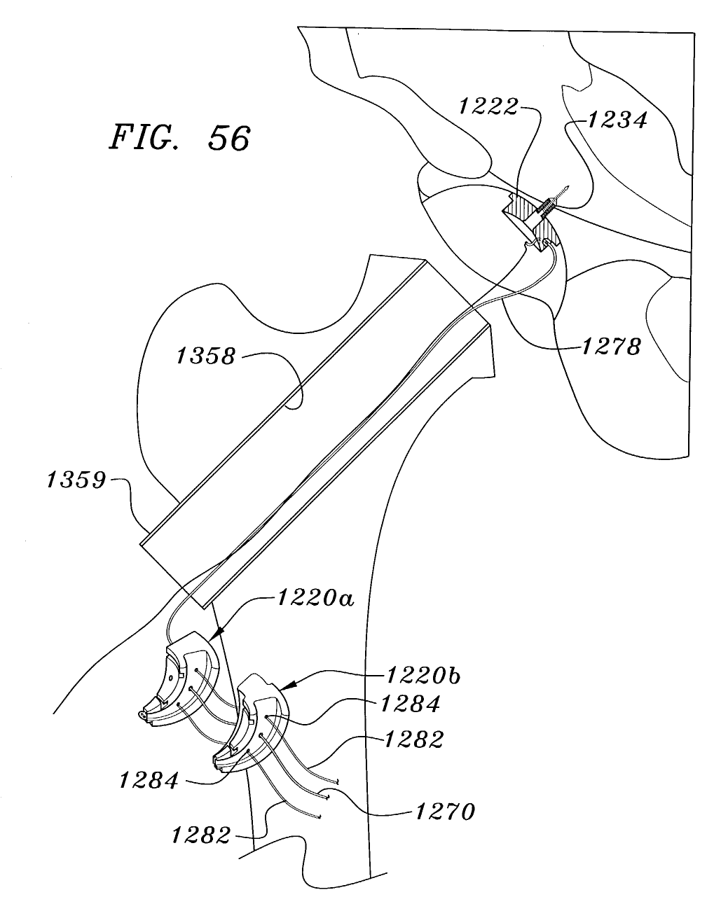

FIGS. 52-56 illustrate another preferred embodiment of the segmented shell, which is indicated as 1216. The segmented shell 1216, which will be mounted into the prepared hip acetabulum is comprised of a plurality of segments 1220a-k that engage one another and a base 1222. The term segment is defined as a separate piece of something and segmented is defined as something that is comprised of separate pieces. Any convenient number of segments can be used; however, eleven segments have been used for illustrative purposes. Therefore the segmented shell 1216 is comprised of separate pieces or segments 920a-k that are joined together with the base 1222 to form the segmented shell 1216. As seen more clearly in FIGS. 52-55, the base 1222 is comprised of a top surface 1224, a bottom surface 1226 and at least one side 1228. The base 1222 has a niche 1230 formed therein that extends into the base 1222 through the side 1228. The base 1222 has a hole 1232 therethrough for receipt of a cannulated screw 1234 for attachment of the base 1222 to the hip bone, as seen in FIG. 56. The bottom surface 1226 of the base 1222 is curved to fit the curvature of the prepared surface on the hip bone.

As seen in FIGS. 52-55, each segment 920a-k has a pair of opposing faces, inner face 1236 and an outer face 1238, a pair of opposing sides, a longitudinally extending first side 1240 and a longitudinally extending second side 1242, a first end 1244 and a second end 1246. The faces 1236 and 1238 and the sides 1240 and 1242 extend between the first end 1244 and the second end 1246. The outer face 1238 of each segment 1220a-k is longitudinally and transversely arcuate, with a curvature that will match the curvature of the prepared surface of the hip bone. At least one of the segments has a hole 1248 that extends through the inner and outer faces 1236 and 1238 of the segment intermediate the first and second ends 1240 and 1242 of the segment. One or more of the holes 1248 may receive a surgical nail (not shown) therethrough, particularly the first segment 1220a, if desired by the surgeon to attach one or more of the segments 920a-k to the prepared bone surface.

The first segment 1220a has a projection formed on and extending outwardly from the first end 1244, which is defined as a tooth 1252. The tooth 1252 has a first end 1254 and a second end 1256, which is attached to the first end 1244 of the segment. In this preferred embodiment, the tooth 1252 is generally straight, as shown in FIG. 53, but is tapered from the first end 1254 toward the second end 1256 as in the embodiment discussed above. The niche 1230 is designed and configured to match the shape of the tooth 1252. The tooth is not angled as the segments are locked and cannot pivot outwardly.

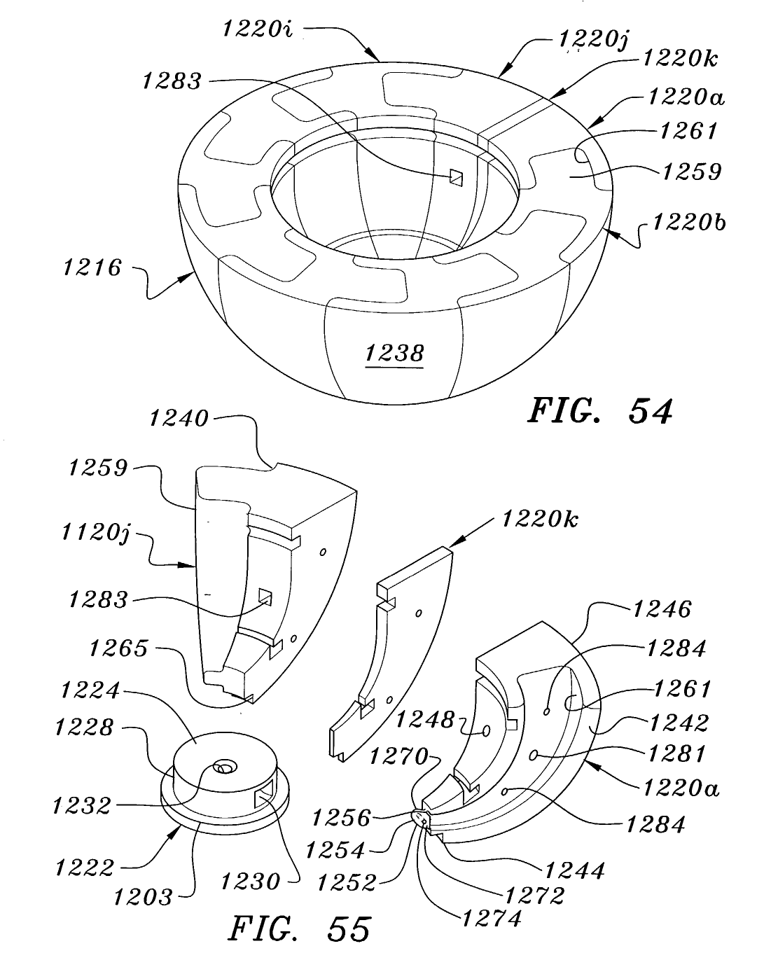

As seen most clearly in FIGS. 52 and 54, all but three segments, segments 1220a, 1220j, and 1220k, of the segments 1220a-k have the same interlocking structure. The segments 920b-i are identical, each having a tongue 1259 that extends outwardly from the first side 1240. A groove 1261 is formed into the second side 1242 of each segment 1220b-i. Therefore, adjacent pairs of segments, for example segment 1220d and segment 1220e, engage one another when the tongue 1259 of the first segment 920d, of the pair of segments, is received in the groove 1261 of the second segment 1220e, of the pair of segments, creating the well-known tongue and groove joint.

Segments 1220a, 1220j, and 1220k identify respectively a first segment, a next-to-last segment, and a last segment. These three segments are structured differently so that the shell of segments may be closed. The first segment 1220a has a groove 1261 formed in the second side 1242 and a and a smooth surface formed on the first side 1240. The next-to-last segment 1220j as a tongue 1259 formed in the first side 1240 and a smooth surface formed on the second side 1242. The last segment 1220k has a smooth surface formed on both sides 1240 and 1242.

In a preferred embodiment, segment 1222k is preferably wedge shaped, with the arc of the outer surface 1238 being narrower than the arc of the inner surface 1236. The adjacent sides of the two adjoining segments. 920a and 920j, are also modified to receive the wedge shape of the segment 920i. This makes the insertion of the last segment into the shell 1216 easier.

The base 1222 has a lip 1263 formed outwardly from the side 1228, which incorporates the bottom surface 1226 of the base 1222. On the underside of the first ends 1244 of each segment a shoulder 1265 is formed thereon which is sized to receive the lip 1263 of the base 1222 therein.

In a preferred embodiment of prosthetic components 1216 is constructed from titanium or cobalt chrome. In other preferred embodiments segmented shell 1216 may be constructed from other materials that are very resistant to wear and are suitable for the purpose.

In a preferred embodiment shell 1216 will be received by the surgeon in disassembled form for thorough disinfecting prior to implantation through assembly in the patient by the surgeon. To assist in the assembly of the shell 16, the tooth 1252 of the first segment 1220a has a hole 1268 passing through the opposing sides 1270 and 1272 of the tooth 1252, creating an opening 1274 in the opposing sides 1270 and 1272 of the tooth 1252. As seen in FIG. 52, the niche 1230 has a pair of conduits 1276 extending from the niche 1230 through the top surface 1224 of the base 1222, so that when the tooth 1252 is inserted within the niche 1230 each opening 1274 is aligned with one of the conduits of the pair of conduits 1276 as seen in FIG. 52. The use of the holes and conduits will be discussed further below during discussion of the method for installation of this preferred embodiment of the joint prosthesis 10.

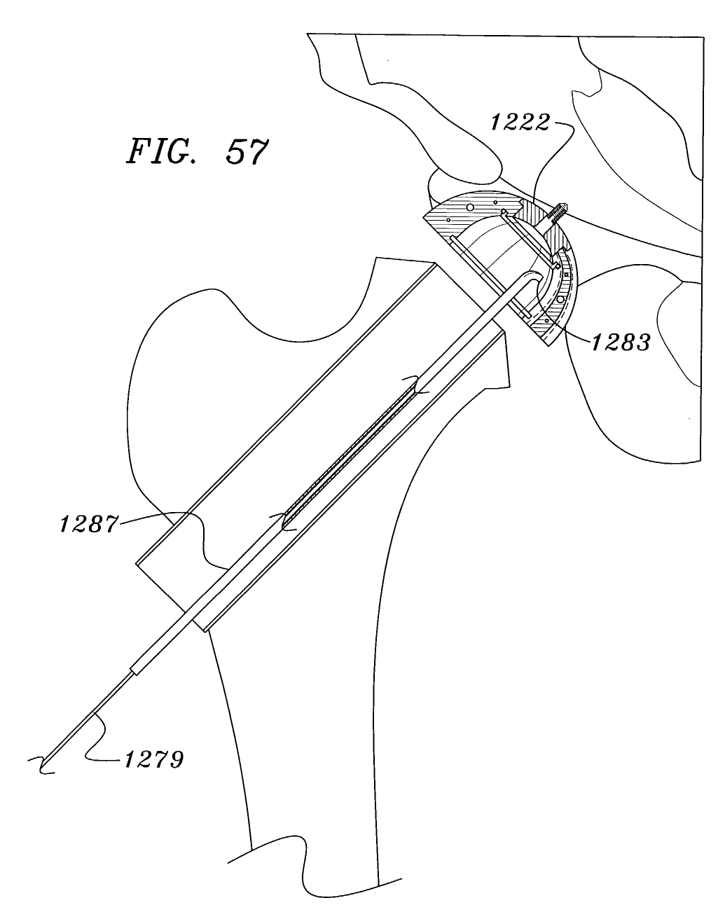

As seen in FIG. 57 the base 1222 has already been implanted in the hip bone with a cannulated screw 1234. This was done by using the same steps in which the base 180 was attached to the hip bone in the first embodiment. That is, by passing the base 1222 through the bore 1358 through the femur which has been lined with a sleeve 1359. The base 1222 is guided in to place by mounting it upon a guide wire that has been embedded in the center of the patient's acetabulum. A cannulated screw 934 was then passed down the same guide wire and through the hole 1232 of the base 1222. The cannulated screw 1234 is then screwed into the bone to firmly hold the base to the hip bone.

As seen in FIG. 56, the first segment 1220a is shown mounted on a closed loop 1278 of very flexible wire. The loop 1278 passes through one conduit of the pair of conduits 1276, through the hole 1268 in the tooth 1252 and then out through the other conduit of the pair of conduits 1276. A tensioning as ire 1279 is attached to the first segment 1220a with a snap off connection. The tensioning wire 1279 is long enough so that it can extend through all the segments and out the sleeve 1359.

The segments are arranged outside the sleeve 1359 until the base is attached to the hip bone. The loop 1278 must be long enough that it extends outwardly from the sleeve 1358 even while the first segment 1220a is outside the sleeve 1358. The loop is pulled causing the first segment 1220a to pass through the sleeve 1359 and polling the tooth 1252 into the niche 1230. Once the tooth has entered the niche in wire is pulled by any well-known tensioning means to obtain the desired insertion force to seat the tooth into the niche. The loop 1278 may then be cut and removed. The next segment 1220b is mounted on the tensioning wire 1279 by passing the tensioning wire through the hole 1281 that is formed in each segment 1220b-i. The segment 1220b is installed so that the tongue 1259 is inserted into groove 1261 of segment 1220a. It may be necessary to assist in the placement of the segments through the use of a long tool that can be inserted through the sleeve and used to adjust the alignment of any errant segment.

In other preferred embodiments, the method for implanting may include guide threads 1282 that are attached to the first segment 1220a. The guide threads 1282 are passed through holes 1284 in each additional segment, so that the segments may be guided into proper alignment with the adjacent segment already installed. As soon as all the segments are loosely aligned with one another these guide threads are removed.

When the next to last segment 1220j is mounted on the tensioning wire 1279, the wire passes out through an access port 1283, as seen in FIGS. 54 and 55, that interconnects with the tensioning wire hole 1281, as seen in FIG. 52, in the front face 1236 of the next to last segment. As seen in FIG. 45, a tensioning tool, conveniently a second tensioner tool 1287, is used to direct the tensioning wire out through the sleeve 1359 so that tension may be applied to the tensioning wire by any well-known tensioning tool that is suitable for the purpose. The application of this tension causes the tongues to be fully inserted within their corresponding grooves. The last segment 1220k is cooled by liquid nitrogen to between −100 and −150 degrees Fahrenheit causing the segment to contract, so that it can be inserted between the next to last segment 1220j and the first segment 1220a. Once the segment warms it expands and applies pressure on the adjacent segments locking the segments together. Additional tension may then be applied to the tensioning wire causing it to be snapped off from the first segment 1220a, permitting the tensioning wire to be removed from the shell 1216.

The segment shell 1216 is now ready for the installation of the ball implant. This segmented shell 1216 will receive the shaft 914 in the same manner as described above.

While the foregoing describes particularly preferred embodiments of the present invention, it is to be understood that numerous variations and modifications of the structure of the prosthetic components and the method for implantation will occur to those skilled in the art. Accordingly, the foregoing description is to be considered illustrative only of the principles of this invention and is not to be considered limitative thereof, the scope of the invention being determined solely by the claims appended hereto.

What is claimed is:

1. A joint prosthesis comprising:
a segmented shell comprising;
a base having a top surface, a bottom surface and at least one side extending therebetween, said base having at least one niche extending into said side of said base;
a plurality of segments, each segment of said plurality of segments, having a first end, a second end, a pair of opposing faces extending between said first end and said second end, and a pair of opposing sides extending between said pair of opposing faces, said first end of at least one segment having a tooth attached thereto and extending outwardly therefrom, said tooth being receivable in said niche in said base, said first end of each of the remaining segments of said plurality of segments engaging said base such that each side of each said segment, of said plurality of segments, is adjacent one of said opposing sides of another one of said plurality of segments, each segment, of said plurality of segments, being longitudinally and transversely arcuate, such that when each segment, of said plurality of segments, is aligned with adjacent segments, of said plurality of segments, said plurality of segments form a cup-shaped segmented shell having an interior surface, an outer face and a longitudinal axis;
means for attaching said segmented shell to a prepared area on the hip bone of a patient;
a cup, said cup having an exterior surface and an interior surface, said exterior surface of said cup being sized and configured to be received in said segmented shell forming a new acetabular socket; and
a shaft having a first end and a second end, said shaft being sized and configured to be receivable in a prepared femur of the patient, and said first end of said shaft having a ball formed thereon that is received by said cup for movement therein.

2 A joint prosthesis as in claim 1, wherein said segmented shell further comprises;
each segment of said plurality of segments, with the exception of a first and a last segment, having a leg extending outwardly from said first side, said leg having a first end angled inwardly toward said longitudinal axis of said shell forming a first channel, said outer face of said each segment having a slot formed therein, said slot and said second side forming an outwardly extending ridge thereon which is radially shortened, such that adjacent pairs of segments engage one another by said first end of said leg of one segment of said pair of segments being received in said slot of said other one of said pair of segments and said channel of said one segment receiving said ridge of said other one of said pair of segments therein;
said first segment having said leg extending outwardly from said first side and a second leg extending outwardly from said second side; and
said last segment having said slot formed in said outer face thereof and a second slot formed in said outer face thereof.

3. A joint prosthesis as in claim 1, wherein said tooth has opposing sides and a hole therethrough passing through said opposing sides creating an opening in each side of said tooth, and said base having a pair of conduits extending from said niche to and through said top surface of said base, such that when said tooth is inserted in said niche, each said opening is aligned with a corresponding one of said pair of conduits in said base; and at least one loop of thread, said loop of thread passing through one of said pair of conduits, through said hole in said tooth and through the other conduit of said pair of conduits in said base, such that pulling on said loop guides said tooth into said niche in said base.

4. A joint prosthesis as in claim 1, comprising a tooth formed on each segment and a plurality of niches formed in said base, each said niche being tapered from said open end inwardly, each tooth being tapered toward said first end of said tooth, a portion of each said niche, that is spaced apart from said open end of said niche, being angled toward said top surface of said base, and a portion of each said tooth proximal said first end of said tooth being angled toward said top surface of said base such that said tooth is fully inserted in said niche.

5. A joint prosthesis as in claim 1, comprising a locking recess formed in said interior surface of said shell and a locking ring, being generally radially expandable between a cooled state and a warmed state, said locking ring being sized and configured to be received in said recess when said ring is in said cooled state;

6. A joint prosthesis as in claim 1;
wherein said shaft further comprises a body and a neck attaching said ball to said body, said body having a bottom surface;
at least one tube having first and second open ends, said tube opening through said bottom surface of said body and extending so that said second end of said tube opens through said neck; and
a plug of self sealing material inserted in said tube proximal to said bottom surface of said body such that said tube is sealed, but said plug being penetrable by a hollow needle, whereby said tube and joint area may be flushed and debris removed from said tube and joint area.

7. A joint prosthesis as in claim 6, said shaft further comprising,
said body having at least one longitudinally extending side wall,
a second tube having a first open end extending through said bottom surface of said shaft and a second closed second end, and
at least one secondary tube passing through said side wall of said body and through said second tube such that said secondary tube is in fluid flow communication with said second tube.

8. A joint prosthesis as in claim 1, wherein said shaft further comprises, a body and a neck attaching said ball to said body, said body having a bottom surface; and
an annular skirt having a first end sealingly attached to said neck and a second end sealingly attached to said cup.

9. A joint prosthesis as in claim 1 further comprising:
said plurality of segments being joined to one another by a tongue and groove joint, each segment, of said plurality of segments, being longitudinally and transversely arcuate, such that when each side of said opposing sides of each said segment, of said plurality of segments, is adjacent one of said opposing sides of another one of said plurality of segments, said plurality of segments define a cup-shaped segmented shell having an interior surface, an exterior surface and a longitudinal axis;

10. A joint prosthesis as in claim 9 wherein said shell further comprises;
each segment of said plurality of segments, with the exception of a first segment, a next to last segment and a last segment, having a tongue extending from one side and a groove formed in the opposing side, said first segment having a tooth attached to and projecting outwardly from said first end of said first segment, said tooth being receivable in said niche in said base, said first side of said first segment being smooth and said second side having a groove formed therein, said next-to-last segment having a tongue extending from said first side and a smooth second side, and said last segment having two smooth opposing sides, said tongue, of each segment of said plurality of segments having a tongue, being sized and configured to be received in the groove of an adjacent segment defining a tongue and groove joint.

11. A joint prosthesis as in claim 9 wherein said shell further comprises;
a tensioning wire; and
said plurality of segments comprising a first segment, a next to last segment, and a plurality of segments therebetween, said first segment having said tensioning wire attached thereto, said tensioning wire extending through a tensioning hole in each one of said plurality of segments therebetween, and said next to last segment having a tensioning hole through a portion of said segment and having a port that extends through said front face to connect with said tensioning hole such that said tensioning wire extends outwardly from said port.

12. A joint prosthesis as in claim 11 wherein said shell further comprises;
said tensioning wire having a first end and a second end, said first end being attached to said first segment such that under a predetermined tension, said tensioning wire disengages from said first segment.

13. A joint prosthesis as in claim 9 wherein said shell further comprises;
an annular recess formed in said inner face of said shell and a lock ring being generally radially expandable between a cooled state and a warmed state, said lock ring being sized and configured to be received in said recess when said lock ring is in said cooled state;

14. A joint prosthesis as in claim 9 wherein said base comprises a lip extending therefrom and each said segment of said plurality of segments having a shoulder formed in the outer face of said first end thereof, such that said shoulder engages said lip of said base.

15. A joint prosthesis as in claim 9;
wherein said shaft further comprises a body and a neck attaching said ball to said body, said body having a bottom surface;
at least one tube having first and second open ends, said tube opening through said bottom surface of said body and extending so that said second end of said tube opens through said neck; and
a plug of self sealing material inserted in said tube proximal to said bottom surface of said body such that said tube is sealed, but said plug being penetrable by a hollow needle, whereby said tube and joint area may be flushed and debris removed from said tube and joint area.

16. A joint prosthesis as in claim 15, said shaft further comprising,
said body having at least one longitudinally extending side wall,
a second tube having a first open end extending through said bottom surface of said shaft and a closed second end, and
at least one secondary tube passing through said side wall of said body and through said second tube such that said secondary tube is in fluid flow communication with said second tube.

17. A joint prosthesis as in claim 1 comprising a layer of polymer cement between said segmented shell and said cup.

18. A method for installing a joint prosthesis, comprising the steps of:
preparing the patient for surgery
aligning the patients body so that a longitudinal axis extending through the neck and head of the femur of the patient passes through the geometric center of the acetabulum of the patient;
making an incision to expose the femur at the point at which the axis exits the femur distal said head of said femur;
inserting a guide wire through said femur, coincident with said longitudinal axis of said joint and into the geometric center of the acetabulum socket of the patient;
using said guide wire to align a drill while boring a hole through said femur and into said hip bone;
removing at least the major portion of said head of said femur and a portion of the acetabulum of said patient;
installing a segmented first shell through the hole in the femur, said first shell comprising a base and a plurality of segments;
inserting the guide wire through a hole in said base and attaching said base to the bone of the hip, leaving the first segment attached to said base by a loop of wire passing through one of a pair of conduits, through a tooth extending from at least one segment and through the other conduit of said pair of conduits formed in said base;
inserting the first segment into said hole through said femur by pulling on the far end of said loop of wire from the segment until said tooth of said segment is seated within said niche formed in said base;
inserting each subsequent segment such that each segment engages said base and the sides of an adjacent segment, until the last segment whose sides engage the sides of the next to last segment and the first segment;
removing said wire loop from said tooth;
inserting a cup through the hole in said femur and mounting said cup in said first shell;
inserting the first end of a shaft into the hole through said femur, said shaft having a ball on said first end of said shaft, said ball being sized and configured to be received by said cup, said shaft being inserted into the hole in the femur until said ball is received by said cup for movement therein; and
closing said incision.

19. A method for installing a joint prosthesis as in 18 comprising the further steps of;
inserting each loop of a plurality of loops through one of said teeth attached to one of said plurality of segments and to a corresponding pair of conduits;
pulling on said loops of wire in succession from said loop attached to said second segment to said loop attached to said last segment such that each said tooth engages said corresponding niche and the sides of the segments adjacent thereto, said last segment engaging the sides of the adjacent segment and the first segment;

20. A method for installing a joint prosthesis as in 19 comprising the further step of;
applying tension to the wire loops attached to said segments, thereby moving said teeth further into said niches.

21. A method for installing a joint prosthesis as in 18 comprising the further step of;
attaching at least one segment to the hip bone of the patient.

22. A method for installing a joint prosthesis as in 18, comprising the further steps of:
mounting said second segment on a tensioning wire extending from said first segment and inserting each segment through said hole in said femur using said tensioning wire to position said segment into engagement with said first segment, said segments being defined as said first and second segments, a next to last segment, intermediate segments between said second segment and said next to last segment, and a last segment;
mounting said intermediate segments on said tensioning wire extending from said first segment and inserting each intermediate segment through said hole in said femur using said tensioning wire to position each segment into engagement with an adjacent segment;
mounting said next to last segment on said tensioning wire such that said tensioning wire extends outwardly from an access port through the inner face of said next to last segment and inserting said next to last segment through said hole in said femur until said next to last segment engages the last previously installed intermediate segment;
tensioning said tensioning wire such that adjacent sides of said segments tightly engage one another;
cooling said last segment and while maintaining said tension in said tension wire, inserting said last segment between said first and next to last segments; and
applying tension on said tensioning wire until said wire is released from said first segment and removed from said segmented shell.

23 A method for installing a joint prosthesis as in 22, comprising the further steps of:
mounting said second segment and each subsequent segment on at least one guide thread extending from said first segment to guide said segments into engagement with the adjacent segment.

24. A joint prosthesis comprising:
a shell comprising a plurality of segments, said shell having a longitudinal axis and an interior surface having an annular recess formed therein;
a ring being generally radially expandable between a cooled state and a heated state, said ring being sized and configured to be received in said recess when in said cooled state;
means for attaching said shell to a prepared area on the hip bone of a patient;
a cup having an exterior surface and an interior surface, said exterior surface of said cup being sized and configured to be received in said shell forming a new acetabular socket; and
a shaft having a first end and a second end, said shaft being sized and configured to be receivable in a prepared femur of the patient, and said first end of said shaft having a ball formed thereon that is received by said cup for movement therein.

25. A joint prosthesis comprising:
a segmented shell,
means for attaching said segmented shell to a prepared area on the hip bone of a patient;
a cup having an exterior surface and an interior surface, said exterior surface of said cup being sized and configured to be received in said segmented shell forming a new acetabular socket; and
a shaft, said shaft comprising a body, a ball and a neck attaching said ball to said body, said body having a bottom surface and said body being sized and configured to be receivable in a prepared femur of the patient, and said ball being received by said cup for movement therein;
at least one tube having first and second open ends, said tube opening through said bottom surface of said body and extending so that said second end of said tube opens through said neck; and
a plug of self sealing material inserted in said tube, whereby said tube is sealed but penetrable by a hollow needle for flushing and removal of debris from said tube and joint area.

26. A joint prosthesis comprising:
a segmented shell,
means for attaching said segmented shell to a prepared area on the hip bone of a patient;
a cup having an exterior surface and an interior surface, said exterior surface of said cup being sized and configured to be received in said segmented shell forming a new acetabular socket; and
a shaft, said shaft comprising a body, a ball and a neck attaching said ball to said body, said body having a bottom surface and said body being sized and configured to be receivable in a prepared femur of the patient, and said ball being received by said cup for movement therein; and
an annular skirt having a first end sealingly attached to said neck and a second end sealingly attached to said cup.
